(19) United States
(12) Patent Application Publication
(10) Pub. No.: US 2016/0230172 A1
(43) Pub. Date: Aug. 11, 2016

(54) COMPOSITIONS FOR MODULATING EXPRESSION OF C9ORF72 ANTISENSE TRANSCRIPT

(71) Applicant: IONIS PHARMACEUTICALS, INC., Carlsbad, CA (US)

(72) Inventor: Frank Rigo, Carlsbad, CA (US)

(73) Assignee: Ionis Pharmaceuticals, Inc., Carlsbad, CA (US)

(21) Appl. No.: 15/029,039

(22) PCT Filed: Oct. 14, 2014
(86) PCT No.: PCT/US14/60512

§ 371 Date: Apr. 13, 2016

Related U.S. Application Data
(60) Provisional application No. 61/890,849, filed on Oct. 14, 2013.

(51) Int. Cl. C12N 15/113

(52) U.S. Cl. CPC C12N 15/113; C12N 2310/113; C12N 2310/315; C12N 2310/3341; C12N 2310/321; C12N 2310/341; C12N 2310/351

(57) ABSTRACT

Disclosed herein are compositions and methods for reducing expression of C90RF72 antisense transcript in an animal with C90RF72 antisense transcript specific inhibitors. Such methods are useful to treat, prevent, or ameliorate neurodegenerative diseases in an individual in need thereof. Such C90RF72 antisense transcript specific inhibitors include antisense compounds.

### SEQUENCE LISTING

The present application is being filed along with a Sequence Listing in electronic format. The Sequence Listing is provided as a file entitled BIOL0236WOSEQ_ST25.txt created Oct. 14, 2014, which is 140 Kb in size. The information in the electronic format of the sequence listing is incorporated herein by reference in its entirety.

### FIELD

Provided are compositions and methods for inhibiting expression of C9ORF72 antisense transcript in an animal. Such compositions and methods are useful to treat, prevent, or ameliorate neurodegenerative diseases, including amyotrophic lateral sclerosis (ALS), frontotemporal dementia (FTD), corticalbasal degeneration syndrome (CBD), atypical Parkinsonian syndrome, and olivopontocerellar degeneration (OPCD).

### BACKGROUND

Amyotrophic lateral sclerosis (ALS) is a fatal neurodegenerative disease characterized clinically by progressive paralysis leading to death from respiratory failure, typically within two to three years of symptom onset (Rowland and Shneider, N. Engl. J. Med., 2001, 344, 1688-1700). ALS is the third most common neurodegenerative disease in the Western world (Hirtz et al., Neurology, 2007, 68, 326-337), and there are currently no effective therapies. Approximately 10% of cases are familial in nature, whereas the bulk of patients diagnosed with the disease are classified as sporadic as they appear to occur randomly throughout the population (Chio et al., Neurology, 2008, 70, 533-537). There is growing recognition, based on clinical, genetic, and epidemiological data, that ALS and frontotemporal dementia (FTD) represent an overlapping continuum of disease, characterized pathologically by the presence of TDP-43 positive inclusions throughout the central nervous system (Lino and Hodges, J. Clin. Neurosci., 2009, 16, 1131-1135; Neumann et al., Science, 2006, 314, 130-133).

To date, a number of genes have been discovered as causative for classical familial ALS, for example, SOD1, TARDBP, FUS, OPTN, and VCP (Johnson et al., Neuron, 2010, 68, 857-864; Kwiatkowski et al., Science, 2009, 323, 1205-1208; Maruyama et al., Nature, 2010, 465, 223-226; Rosen et al., Nature, 1993, 362, 59-62; Sreedharan et al., Science, 2008, 319, 1668-1672; Vance et al., Brain, 2009, 129, 868-876). Recently, linkage analysis of kindreds involving multiple cases of ALS, FTD, and ALS-FTD had suggested that there was an important locus for the disease on the short arm of chromosome 9 (Boxer et al., J. Neurol. Neurosurg. Psychiatry, 2011, 82, 196-203; Morita et al., Neurology, 2006, 66, 839-844; Pearson et al. J. Nerol., 2011, 258, 647-655; Vance et al., Brain, 2006, 129, 868-876). This mutation has been found to be the most common genetic cause of ALS and FTD. It is postulated that the ALS-FTD causing mutation is a large hexanucleotide (GGGGCC) repeat expansion in the first intron of the C9ORF72 gene (Renton et al., Neuron, 2011, 72, 257-268; DeJesus-Hernandez et al., Neuron, 2011, 72, 245-256). A founder haplotype, covering the C9ORF72 gene, is present in the majority of cases linked to this region (Renton et al., Neuron, 2011, 72, 257-268). This locus on chromosome 9p21 accounts for nearly half of familial ALS and nearly one-quarter of all ALS cases in a cohort of 405 Finnish patients (Laaksovirta et al, Lancet Neurol., 2010, 9, 978-985).

There are currently no effective therapies to treat such neurodegenerative diseases. Therefore, it is an object to provide compositions and methods for the treatment of such neurodegenerative diseases.

### SUMMARY

Provided herein are compositions and methods for modulating levels of C9ORF72 antisense transcript in cells, tissues, and animals. In certain embodiments, C9ORF72 antisense transcript specific inhibitors modulate expression of C9ORF72 antisense transcript. In certain embodiments, C9ORF72 antisense transcript specific inhibitors are nucleic acids, proteins, or small molecules.

In certain embodiments, modulation can occur in a cell or tissue. In certain embodiments, the cell or tissue is in an animal. In certain embodiments, the animal is a human. In certain embodiments, C9ORF72 antisense transcript levels are reduced. In certain embodiments, C9ORF72 antisense transcript associated RAN translation products are reduced. In certain embodiments, the C9ORF72 antisense transcript associated RAN translation products are poly-(proline-alanine), poly-(proline-arginine), and poly-(proline-glycine). In certain embodiments, the C9ORF72 antisense transcript contains a hexanucleotide repeat expansion. In certain embodiments, the hexanucleotide repeat is transcribed in the antisense direction from the C9ORF72 gene. In certain embodiments, the hexanucleotide repeat expansion is associated with a C9ORF72 associated disease. In certain embodiments, the hexanucleotide repeat expansion is associated with a C9ORF72 hexanucleotide repeat expansion associated disease. In certain embodiments, the hexanucleotide repeat expansion comprises at least 24 GGCCCC, CCCCCC, GCCCCC, and/or CGCCCC repeats. In certain embodiments, the hexanucleotide repeat expansion is associated with nuclear foci. In certain embodiments, C9ORF72 antisense transcript associated RAN translation products are associated with nuclear foci. In certain embodiments, the antisense transcript associated RAN translation products are poly-(proline-alanine) and/or poly-(proline-arginine). In certain embodiments, the compositions and methods described herein are useful for reducing C9ORF72 antisense transcript levels, C9ORF72 antisense transcript associated RAN translation products, and nuclear foci. Such reduction can occur in a time-dependent manner or in a dose-dependent manner.

Also provided are methods useful for preventing, treating, ameliorating, and slowing progression of diseases and conditions associated with C9ORF72. In certain embodiments, such diseases and conditions associated with C9ORF72 are neurodegenerative diseases. In certain embodiments, the neurodegenerative disease is amyotrophic lateral sclerosis (ALS), frontotemporal dementia (FTD), corticalbasal degeneration syndrome (CBD), atypical Parkinsonian syndrome, or olivopontocerellar degeneration (OPCD).

Such diseases and conditions can have one or more risk factors, causes, or outcomes in common. Certain risk factors and causes for development of a neurodegenerative disease, and, in particular, ALS and FTD, include genetic predisposition and older age.

In certain embodiments, methods of treatment include administering a C9ORF72 antisense transcript specific inhibitor to an individual in need thereof. In certain embodiments, the C9ORF72 antisense transcript specific inhibitor is a nucleic acid. In certain embodiments, the nucleic acid is an antisense compound. In certain embodiments, the antisense compound is an antisense oligonucleotide. In certain embodiments, the antisense oligonucleotide is complementary to a C9ORF72 antisense transcript. In certain embodiments, the antisense oligonucleotide is a modified antisense oligonucleotide.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure herein are hereby expressly incorporated by reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:

“2′-O-methoxyethyl” (also 2′-MOE and 2′-OCH₂CH₂—OCH₃ and MOE) refers to an O-methoxy-ethyl modification of the 2′ position of a furanose ring. A 2′-O-methoxyethyl modified sugar is a modified sugar.

“2′-MOE nucleoside” (also 2′-O-methoxyethyl nucleoside) means a nucleoside comprising a MOE modified sugar moiety.

“2′-substituted nucleoside” means a nucleoside comprising a substituent at the 2′-position of the furanose ring other than H or OH. In certain embodiments, 2′-substituted nucleosides include nucleosides with bicyclic sugar modifications.

“5-methylcytosine” means a cytosine modified with a methyl group attached to the 5′ position. A 5-methylcytosine is a modified nucleobase.

“About” means within ±7% of a value. For example, if it is stated, “the compounds affected at least about 70% inhibition of C9ORF72 antisense transcript”, it is implied that the C9ORF72 antisense transcript levels are inhibited within a range of 63% and 77%.

“Administered concomitantly” refers to the co-administration of two pharmaceutical agents in any manner in which the pharmacological effects of both are manifest in the patient at the same time. Concomitant administration does not require that both pharmaceutical agents be administered in a single pharmaceutical composition, in the same dosage form, or by the same route of administration. The effects of both pharmaceutical agents need not manifest themselves at the same time. The effects need only be overlapping for a period of time and need not be coextensive.

“Administering” means providing a pharmaceutical agent to an animal, and includes, but is not limited to administering by a medical professional and self-administering.

“Amelioration” refers to a lessening, slowing, stopping, or reversing of at least one indicator of the severity of a condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein product encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels or in the absence of the antisense compound.

“Antisense mechanisms” are all those mechanisms involving hybridization of a compound with a target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“C9ORF72 antisense transcript” means transcripts produced from the non-coding strand (also antisense strand and template strand) of the C9ORF72 gene. The C9ORF72 antisense transcript differs from the canonically transcribed “C9ORF72 sense transcript”, which is produced from the coding strand (also sense strand) of the C9ORF72 gene.

“C9ORF72 antisense transcript associated RAN translation products” means aberrant peptide or di-peptide polymers translated through RAN translation (i.e., repeat-associated, and non-ATG-dependent translation). In certain embodiments, the C9ORF72 antisense transcript associated RAN translation products are any of poly-(proline-alanine), poly-(proline-arginine), and poly-(proline-glycine).

“C9ORF72 antisense transcript specific inhibitor” refers to any agent capable of specifically inhibiting the expression of C9ORF72 antisense transcript and/or its expression products at the molecular level. For example, C9ORF72 specific antisense transcript inhibitors include nucleic acids (including antisense compounds), siRNAs, aptamers, antibodies, peptides, small molecules, and other agents capable of inhibiting the expression of C9ORF72 antisense transcript and/or its expression products, such as C9ORF72 antisense transcript associated RAN translation products.

“C9ORF72 associated disease” means any disease associated with any C9ORF72 nucleic acid or expression product thereof, regardless of which DNA strand the C9ORF72 nucleic acid or expression product thereof is derived from. Such diseases may include a neurodegenerative disease. Such neurodegenerative diseases may include ALS and FTD.

“C9ORF72 foci” means nuclear foci comprising a C9ORF72 transcript. In certain embodiments, a C9ORF72 foci comprises at least one C9ORF72 sense transcript (herein “C9ORF72 sense foci”). In certain embodiments, C9ORF72 sense foci comprise C9ORF72 sense transcripts comprising any of the following hexanucleotide repeats: GGGGCC, GGGGGG, GGGGGC, and/or GGGGCG. In certain embodiments, a C9ORF72 foci comprises at least one C9ORF72 antisense transcript (herein “C9ORF72 antisense foci”). In certain embodiments, C9ORF72 antisense foci comprise C9ORF72 antisense transcripts comprising any of the following hexanucleotide repeats: GGCCCC, CCCCCC, GCCCCC, and/or CGCCCC. In certain embodiments, C9ORF72 foci comprise both C9ORF72 sense transcripts and C9ORF72 antisense transcripts.

“C9ORF72 hexanucleotide repeat expansion associated disease” means any disease associated with a C9ORF72 nucleic acid containing a hexanucleotide repeat expansion. In certain embodiments, the hexanucleotide repeat expansion may comprise any of the following hexanucleotide repeats: GGGGCC, GGGGGG, GGGGGC, GGGGCG, GGCCCC, CCCCCC, GCCCCC, and/or CGCCCC. In certain embodiments, the hexanucleotide repeat is repeated at least 24 times. Such diseases may include a neurodegenerative disease. Such neurodegenerative diseases may include ALS and FTD.

“C9ORF72 nucleic acid” means any nucleic acid derived from the C9ORF72 locus, regardless of which DNA strand the C9ORF72 nucleic acid is derived from. In certain embodiments, a C9ORF72 nucleic acid includes a DNA sequence encoding C9ORF72, an RNA sequence transcribed from DNA encoding C9ORF72 including genomic DNA comprising introns and exons (i.e., pre-mRNA), and an mRNA sequence encoding C9ORF72. “C9ORF72 mRNA” means an mRNA encoding a C9ORF72 protein. In certain embodiments, a C9ORF72 nucleic acid includes transcripts produced from the coding strand of the C9ORF72 gene. C9ORF72 sense transcripts are examples of C9ORF72 nucleic acids. In certain embodiments, a C9ORF72 nucleic acid includes transcripts produced from the non-coding strand of the C9ORF72 gene. C9ORF72 antisense transcripts are examples of C9ORF72 nucleic acids.

“C9ORF72 pathogenic associated mRNA variant” means the C9ORF72 mRNA variant processed from a C9ORF72 pre-mRNA variant containing the hexanucleotide repeat. A C9ORF72 pre-mRNA contains the hexanucleotide repeat when transcription of the pre-mRNA begins in the region from the start site of exon 1A to the start site of exon 1B, e.g., nucleotides 1107 to 1520 of the genomic sequence (SEQ ID NO: 2, the complement of GENBANK Accession No. NT_008413.18 truncated from nucleosides 27535000 to 27565000). In certain embodiments, the level of a C9ORF72 pathogenic associated mRNA variant is measured to determine the level of a C9ORF72 pre-mRNA containing the hexanucleotide repeat in a sample.

“C9ORF72 transcript” means an RNA transcribed from C9ORF72. In certain embodiments, a C9ORF72 transcript is a C9ORF72 sense transcript. In certain embodiments, a C9ORF72 transcript is a C9ORF72 antisense transcript.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleosides is chemically distinct from a region having nucleosides without 2′-O-methoxyethyl modifications.

“Chimeric antisense compound” means an antisense compound that has at least two chemically distinct regions, each position having a plurality of subunits.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Comprise,” “comprises,” and “comprising” will be understood to imply the inclusion of a stated step or element or group of steps or elements but not the exclusion of any other step or element or group of steps or elements.

“Contiguous nucleobases” means nucleobases immediately adjacent to each other.

“Designing” or“designed to” refer to the process of designing an oligomeric compound that specifically hybridizes with a selected nucleic acid molecule.

“Diluent” means an ingredient in a composition that lacks pharmacological activity, but is pharmaceutically necessary or desirable. For example, in drugs that are injected, the diluent may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” in the context of modulating an activity or of treating or preventing a condition means the administration of that amount of pharmaceutical agent to a subject in need of such modulation, treatment, or prophylaxis, either in a single dose or as part of a series, that is effective for modulation of that effect, or for treatment or prophylaxis or improvement of that condition. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information, regardless of which DNA strand the coded information is derived from, is converted into structures present and operating in a cell. Such structures include, but are not limited to the products of transcription and translation, including RAN translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” and the external regions may be referred to as the “wings.”

“Gap-narrowed” means a chimeric antisense compound having a gap segment of 9 or fewer contiguous 2′-deoxyribonucleosides positioned between and immediately adjacent to 5′ and 3′ wing segments having from 1 to 6 nucleosides.

“Gap-widened” means a chimeric antisense compound having a gap segment of 12 or more contiguous 2′-deoxyribonucleosides positioned between and immediately adjacent to 5′ and 3′ wing segments having from 1 to 6 nucleosides.

“Hexanucleotide repeat expansion” means a series of six bases (for example, GGGGCC, GGGGGG, GGGGGC, GGGGCG, GGCCCC, CCCCCC, GCCCCC, and/or CGCCCC) repeated at least twice. In certain embodiments, the hexanucleotide repeat expansion may be located in intron 1 of a C9ORF72 nucleic acid. In certain embodiments, the hexanucleotide repeat may be transcribed in the antisense direction from the C9ORF72 gene. In certain embodiments, a pathogenic hexanucleotide repeat expansion includes at least 24 repeats of GGGGCC, GGGGGG, GGGGGC, GGGGCG, GGCCCC, CCCCCC, GCCCCC, and/or CGCCCC in a C9ORF72 nucleic acid and is associated with disease. In certain embodiments, the repeats are consecutive. In certain embodiments, the repeats are interrupted by 1 or more nucleobases. In certain embodiments, a wild-type hexanucleotide repeat expansion includes 23 or fewer repeats of GGGGCC, GGGGGG, GGGGGC, GGGGCG, GGCCCC, CCCCCC, GCCCCC, and/or CGCCCC in a C9ORF72 nucleic acid. In certain embodiments, the repeats are consecutive. In certain embodiments, the repeats are interrupted by 1 or more nucleobases.

“Hybridization” means the annealing of complementary nucleic acid molecules. In certain embodiments, complementary nucleic acid molecules include, but are not limited to, an antisense compound and a target nucleic acid. In certain embodiments, complementary nucleic acid molecules include, but are not limited to, an antisense oligonucleotide and a nucleic acid target.

“Identifying an animal having a C9ORF72 associated disease” means identifying an animal having been diagnosed with a C9ORF72 associated disease or predisposed to develop a C9ORF72 associated disease. Individuals predisposed to develop a C9ORF72 associated disease include those having one or more risk factors for developing a C9ORF72 associated disease, including, having a personal or family history or genetic predisposition of one or more C9ORF72 associated diseases. In certain embodiments, the C9ORF72 associated disease is a C9ORF72 hexanucleotide repeat expansion associated disease. Such identification may be accomplished by any method including evaluating an individual's medical history and standard clinical tests or assessments, such as genetic testing.

“Immediately adjacent” means there are no intervening elements between the immediately adjacent elements.

“Individual” means a human or non-human animal selected for treatment or therapy.

“Inhibiting expression of a C9ORF72 antisense transcript” means reducing the level or expression of a C9ORF72 antisense transcript and/or its expression products (e.g., RAN translation products). In certain embodiments, C9ORF72 antisense transcripts are inhibited in the presence of an antisense compound targeting a C9ORF72 antisense transcript, including an antisense oligonucleotide targeting a C9ORF72 antisense transcript, as compared to expression of C9ORF72 antisense transcript levels in the absence of a C9ORF72 antisense compound, such as an antisense oligonucleotide.

“Inhibiting expression of a C9ORF72 sense transcript” means reducing the level or expression of a C9ORF72 sense transcript and/or its expression products (e.g., a C9ORF72 mRNA and/or protein). In certain embodiments, C9ORF72 sense transcripts are inhibited in the presence of an antisense compound targeting a C9ORF72 sense transcript, including an antisense oligonucleotide targeting a C9ORF72 sense transcript, as compared to expression of C9ORF72 sense transcript levels in the absence of a C9ORF72 antisense compound, such as an antisense oligonucleotide.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Locked nucleic acid” or “LNA” or “LNA nucleosides” means nucleic acid monomers having a bridge connecting two carbon atoms between the 4′ and 2′ position of the nucleoside sugar unit, thereby forming a bicyclic sugar. Examples of such bicyclic sugar include, but are not limited to A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

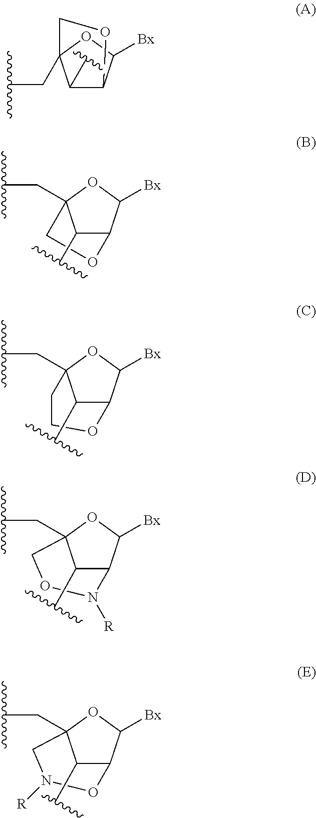

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′- bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore; in the case of the bicylic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, and/or modified nucleobase.

“Modified oligonucleotide” means an oligonucleotide comprising at least one modified internucleoside linkage, modified sugar, and/or modified nucleobase.

“Modified sugar” means substitution and/or any change from a natural sugar moiety.

“Monomer” means a single unit of an oligomer. Monomers include, but are not limited to, nucleosides and nucleotides, whether naturally occurring or modified.

“Motif” means the pattern of unmodified and modified nucleoside in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Nucleotide” means a nucleoside having a phosphate group covalently linked to the sugar portion of the nucleoside.

“Off-target effect” refers to an unwanted or deleterious biological effect associated with modulation of RNA or protein expression of a gene other than the intended target nucleic acid.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, peptide refers to polypeptides and proteins.

“Pharmaceutical agent” means a substance that provides a therapeutic benefit when administered to an individual. In certain embodiments, an antisense oligonucleotide targeted to C9ORF72 sense transcript is a pharmaceutical agent. In certain embodiments, an antisense oligonucleotide targeted to C9ORF72 antisense transcript is a pharmaceutical agent.

“Pharmaceutical composition” means a mixture of substances suitable for administering to as subject. For example, a pharmaceutical composition may comprise an antisense oliognucleotide and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disease, disorder, or condition for a period of time from minutes to days, weeks to months, or indefinitely.

“Prodrug” means a therapeutic agent that is prepared in an inactive form that is converted to an active form within the body or cells thereof by the action of endogenous enzymes or other chemicals or conditions.

“Prophylactically effective amount” refers to an amount of a pharmaceutical agent that provides a prophylactic or preventative benefit to an animal.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Salts” mean a physiologically and pharmaceutically acceptable salts of antisense compounds, i.e., salts that retain the desired biological activity of the parent oligonucleotide and do not impart undesired toxicological effects thereto.

“Segments” are defined as smaller or sub-portions of regions within a target nucleic acid.

“Shortened” or “truncated” versions of antisense oligonucleotides taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies.

“Single-stranded oligonucleotide” means an oligonucleotide which is not hybridized to a complementary strand.

“Sites,” as used herein, are defined as unique nucleobase positions within a target nucleic acid.

“Slows progression” means decrease in the development of the disease.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Stringent hybridization conditions” or “stringent conditions” refer to conditions under which an oligomeric compound will hybridize to its target sequence, but to a minimal number of other sequences.

“Subject” means a human or non-human animal selected for treatment or therapy.

“Targeting” or “targeted” means the process of design and selection of an antisense compound that will specifically hybridize to a target nucleic acid and induce a desired effect.

“Target nucleic acid,” “target RNA,” and “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” or “treating” or “treatment” means administering a composition to effect an alteration or improvement of a disease or condition.

“Unmodified nucleobases” means the purine bases adenine (A) and guanine (G), and the pyrimidine bases (T), cytosine (C), and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Wing segment” means a plurality of nucleosides modified to impart to an oligonucleotide properties such as enhanced inhibitory activity, increased binding affinity for a target nucleic acid, or resistance to degradation by in vivo nucleases.

### Certain Embodiments

Provided herein are compounds comprising a C9ORF72 antisense transcript specific inhibitor.

In certain embodiments, the C9ORF72 antisense transcript specific inhibitor is a C9ORF72 antisense transcript specific antisense compound.

In certain embodiments, the C9ORF72 antisense transcript specific antisense compound comprises an antisense oligonucleotide.

In certain embodiments, the antisense oligonucleotide consists of 12-30 linked nucleosides.

In certain embodiments, the antisense oligonucleotide consists of 16-25 linked nucleosides.

In certain embodiments, the antisense oligonucleotide consists of 18-22 linked nucleosides

In certain embodiments, the antisense oligonucleotide has a nucleobase sequence that is at least 80%, at least 85%, at least 90%, at least 91%, at least 92%, at least 93%, at least 94%, at least 95%, at least 96%, at least 97%, at least 98%, at least 99%, or 100% complementary to a C9ORF72 antisense transcript.

In certain embodiments, the antisense oligonucleotide has a nucleobase sequence that is at least 90% complementary to a C9ORF72 antisense transcript.

In certain embodiments, the antisense oligonucleotide has a nucleobase sequence that is at least 95% complementary to a C9ORF72 antisense transcript.

In certain embodiments, the antisense oligonucleotide has a nucleobase sequence that is 100% complementary to a C9ORF72 antisense transcript.

In certain embodiments, the C9ORF72 antisense transcript is SEQ ID NO: 11.

In certain embodiments, the antisense oligonucleotide has a nucleobase sequence comprising at least 8, at least 9, at least 10, at least 11, at least 12, at least 13, at least 14, at least 15, at least 16, at least 17, at least 18, at least 19, or 20 contiguous nucleobases of a sequence selected from among SEQ ID NO: 19-20, 22-26, 28-32, 34-42, 44, 46-55, 58-59, and 61.

In certain embodiments, the antisense oligonucleotide is a modified antisense oligonucleotide.

In certain embodiments, the modified antisense oligonucleotide comprises at least one modified internucleoside linkage.

In certain embodiments, the at least one modified internucleoside linkage is a phosphorothioate internucleoside linkage.

In certain embodiments, each modified internucleoside linkage is a phosphorothioate internucleoside linkage.

In certain embodiments, the modified antisense oligonucleotide comprises at least one phosphodiester internucleoside linkage.

In certain embodiments, the at least one nucleoside of the modified antisense oligonucleotide comprises a modified nucleobase.

In certain embodiments, the modified nucleobase is a 5-methylcytosine.

In certain embodiments, the at least one nucleoside of the modified antisense oligonucleotide comprises a modified sugar.

In certain embodiments, the at least one modified sugar is a bicyclic sugar.

In certain embodiments, the bicyclic sugar comprises a chemical bridge between the 2′ and 4′ position of the sugar, wherein the chemical bridge is selected from: 4′-CH₂—O-2′; 4′-CH(CH₃)—O-2′; 4′-(CH₂)₂—O-2′; and 4′-CH₂—N(R)—O-2′ wherein R is, independently, H, C₁-C₁₂ alkyl, or a protecting group.

In certain embodiments, the at least one modified sugar comprises a 2′-O-methoxyethyl group.

In certain embodiments, the antisense oligonucleotide is a gapmer.

In certain embodiments, the C9ORF72 antisense transcript specific antisense compound comprises at least one conjugate.

In certain embodiments, the C9ORF72 antisense transcript specific antisense compound consists of an antisense oligonucleotide.

Provided herein are pharmaceutical compositions comprising any compound described herein and a pharmaceutically acceptable diluent or carrier.

Provided herein are pharmaceutical compositions comprising a C9ORF72 antisense transcript specific inhibitior.

Provided herein are pharmaceutical compositions comprising a C9ORF72 antisense transcript specific inhibitor and a C9ORF sense transcript specific inhibitior.

In certain embodiments, the C9ORF72 sense transcript specific inhibitor is a C9ORF72 sense transcript specific antisense compound.

In certain embodiments, the C9ORF72 antisense transcript specific inhibitor is a C9ORF72 antisense transcript specific antisense compound.

In certain embodiments, the C9ORF72 sense transcript specific antisense compound is an antisense oligonucleotide.

In certain embodiments, the C9ORF72 antisense transcript specific antisense compound is an antisense oligonucleotide.

In certain embodiments, the sense transcript is any of SEQ ID NO: 1-10.

Provided herein are uses of any compound described herein for the manufacture of a medicament for treating a neurodegenerative disease.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a C9ORF72 nucleic acid is 12 to 30 subunits in length. In other words, such antisense compounds are from 12 to 30 linked subunits. In certain embodiments, the antisense compound is 8 to 80, 12 to 50, 15 to 30, 18 to 24, 19 to 22, or 20 linked subunits. In certain embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleosides.

In certain embodiments antisense oligonucleotides targeted to a C9ORF72 nucleic acid may be shortened or truncated. For example, a single subunit may be deleted from the 5′ end (5′ truncation), or alternatively from the 3′ end (3′ truncation). A shortened or truncated antisense compound targeted to a C9ORF72 nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a C9ORF72 nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)n-O-2′ bridge, where n=1 or n=2 and 4′-CH₂—O—CH₂-2′). Preferably, each distinct region comprises uniform sugar moieties. The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers described herein include, but are not limited to, for example 5-10-5, 5-10-4, 4-10-4, 4-10-3, 3-10-3, 2-10-2, 5-9-5, 5-9-4, 4-9-5, 5-8-5, 5-8-4, 4-8-5, 5-7-5, 4-7-5, 5-7-4, or 4-7-4.

In certain embodiments, the antisense compound has a “wingmer” motif, having a wing-gap or gap-wing configuration, i.e. an X—Y or Y—Z configuration as described above for the gapmer configuration. Thus, wingmer configurations described herein include, but are not limited to, for example 5-10, 8-4, 4-12, 12-4, 3-14, 16-2, 18-1, 10-3, 2-10, 1-10, 8-2, 2-13, 5-13, 5-8, or 6-8.

In certain embodiments, an antisense compound targeted to a C9ORF72 nucleic acid has a gap-narrowed motif. In certain embodiments, a gap-narrowed antisense oligonucleotide targeted to a C9ORF72 nucleic acid has a gap segment of 9, 8, 7, or 6 2′-deoxynucleotides positioned immediately adjacent to and between wing segments of 5, 4, 3, 2, or 1 chemically modified nucleosides. In certain embodiments, the chemical modification comprises a bicyclic sugar. In certain embodiments, the bicyclic sugar comprises a 4′ to 2′ bridge selected from among: 4′-(CH₂)ₙ-0-2′ bridge, wherein n is 1 or 2; and 4′-CH₂—O—CH₂-2′. In certain embodiments, the bicyclic sugar is comprises a 4′-CH(CH₃)—O-2′ bridge. In certain embodiments, the chemical modification comprises a non-bicyclic 2′-modified sugar moiety. In certain embodiments, the non-bicyclic 2′-modified sugar moiety comprises a 2′-O-methylethyl group or a 2′-O-methyl group.

### Target Nucleic Acids, Target Regions and Nucleotide Sequences

Nucleotide sequences that encode C9ORF72 include, without limitation, the following: the complement of GENBANK Accession No. NM_001256054.1 (incorporated herein as SEQ ID NO: 1), GENBANK Accession No. NT_008413.18 truncated from nucleobase 27535000 to 27565000 (incorporated herein as SEQ ID NO: 2), GENBANK Accession No. BQ068108.1 (incorporated herein as SEQ ID NO: 3), GENBANK Accession No. NM_018325.3 (incorporated herein as SEQ ID NO: 4), GENBANK Accession No. DN993522.1 (incorporated herein as SEQ ID NO: 5), GENBANK Accession No. NM_145005.5 (incorporated herein as SEQ ID NO: 6), GENBANK Accession No. DB079375.1 (incorporated herein as SEQ ID NO: 7), GENBANK Accession No. BU194591.1 (incorporated herein as SEQ ID NO: 8), Sequence Identifier 4141_014_A (incorporated herein as SEQ ID NO: 9), and Sequence Identifier 4008_73_A (incorporated herein as SEQ ID NO: 10).

Nucleotide sequences that encode the C9ORF72 antisense transcript include, without limitation, the following: SEQ ID NO: 11 is a sequence that is complementary to nucleotides 1159 to 1734 of SEQ ID NO: 2 (the complement of GENBANK Accession No. NT_008413.18 truncated from nucleotides 27535000 to 27565000).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for C9ORF72 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein. Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within a target region. In certain embodiments, reductions in C9ORF72 mRNA levels are indicative of inhibition of C9ORF72 expression. Reductions in levels of a C9ORF72 protein are also indicative of inhibition of target mRNA expression. Reduction in the presence of expanded C9ORF72 RNA foci are indicative of inhibition of C9ORF72 expression. Further, phenotypic changes are indicative of inhibition of C9ORF72 expression. For example, improved motor function and respiration may be indicative of inhibition of C9ORF72 expression.

### Hybridization

In some embodiments, hybridization occurs between an antisense compound disclosed herein and a C9ORF72 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a C9ORF72 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a C9ORF72 nucleic acid).

Non-complementary nucleobases between an antisense compound and a C9ORF72 nucleic acid may be tolerated provided that the antisense compound remains able to specifically hybridize to a target nucleic acid. Moreover, an antisense compound may hybridize over one or more segments of a C9ORF72 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a C9ORF72 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a C9ORF72 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a C9ORF72 nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a C9ORF72 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds, are complementary to at least an 11 nucleobase portion of a target segment. In certain embodiments, the antisense compounds, are complementary to at least a 12 nucleobase portion of a target segment. In certain embodiments, the antisense compounds, are complementary to at least a 13 nucleobase portion of a target segment. In certain embodiments, the antisense compounds, are complementary to at least a 14 nucleobase portion of a target segment. In certain embodiments, the antisense compounds, are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a C9ORF72 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage. In certain embodiments, the antisense compounds targeted to a C9ORF72 nucleic acid comprise at least one phosphodiester linkage and at least one phosphorothioate linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. patent Ser. Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is—[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

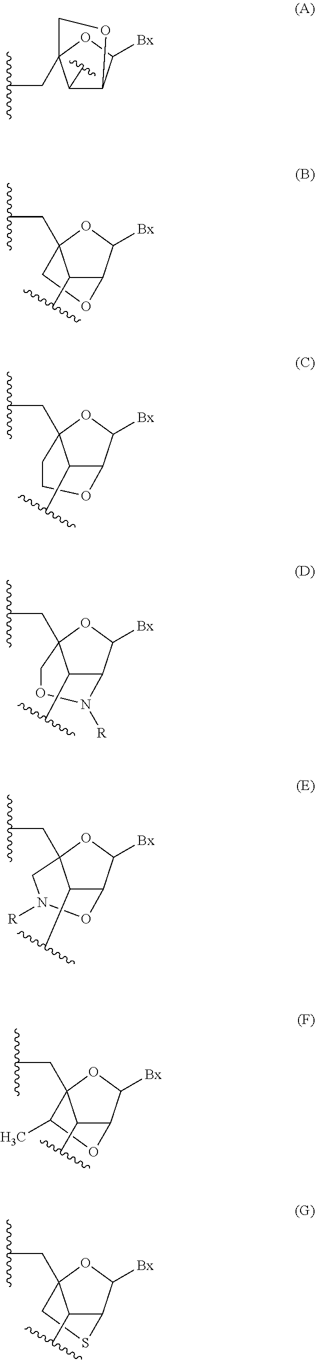

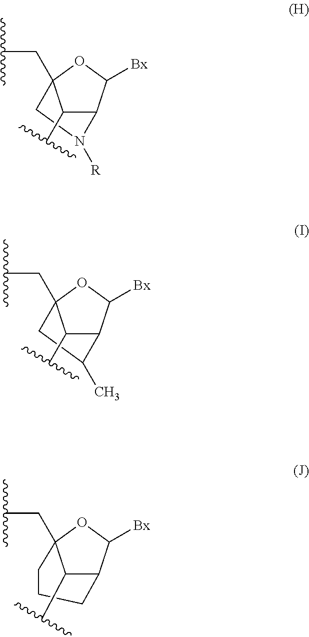

wherein Bx is the base moiety and R is independently H, a protecting group or C₁-C₁₂ alkyl.

In certain embodiments, bicyclic nucleosides are provided having Formula I:

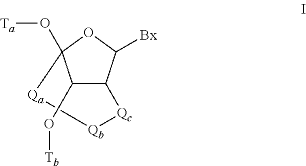

wherein:

Bx is a heterocyclic base moiety;

-Qₐ-Q_{b}-Q_{c}- is —CH₂—N(R_{c})—CH₂—, —C(═O)—N(R_{c})—CH₂-, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

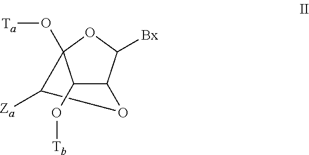

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

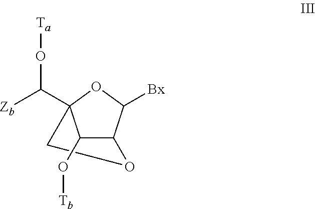

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

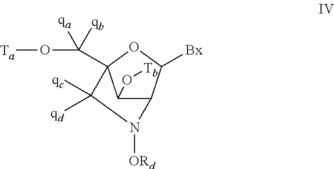

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

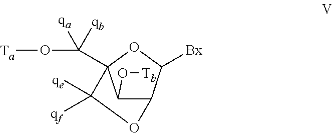

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

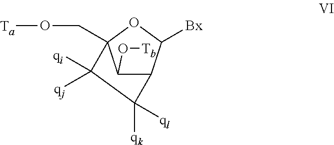

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854), fluoro HNA (F-HNA) or those compounds having Formula VII:

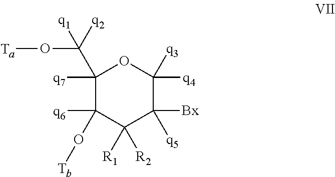

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is H and R₂ is methoxyethoxy.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854).

Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a C9ORF72 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a C9ORF72 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of C9ORF72 nucleic acids can be tested in vitro in a variety of cell types. Cell types used for such analyses are available from commercial vendors (e.g. American Type Culture Collection, Manassas, Va.; Zen-Bio, Inc., Research Triangle Park, N.C.; Clonetics Corporation, Walkersville, Md.) and are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, Calif.). Antisense oligonucleotides are mixed with LIPOFECTIN in OPTI-MEM 1 (Invitrogen, Carlsbad, Calif.) to achieve the desired final concentration of antisense oligonucleotide and a LIPOFECTIN concentration that typically ranges 2 to 12 ug/mL per 100 nM antisense oligonucleotide.

Another reagent used to introduce antisense oligonucleotides into cultured cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a C9ORF72 nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invetrogen, Inc. Eugene, Oreg.). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a C9ORF72 nucleic acid. Methods for designing real-time PCR probes and primers are well known in the art, and may include the use of software such as PRIMER EXPRESS Software (Applied Biosystems, Foster City, Calif.).

### Strand Specific Semi-Quantitative PCR Analysis of Target RNA Levels

Analysis of specific, low abundance target RNA strand levels may be accomplished by reverse transcription, PCR, and gel densitometry analysis using the Gel Logic 200 Imaging System and Kodak MI software (Kodak Scientific Imaging Systems, Rochester, N.Y., USA) according to manufacturer's instructions.

RT-PCR reactions are carried out as taught in Ladd, P. D., et al, (Human Molecular Genetics, 2007, 16, 3174-3187) and in Sopher, B. L., et al, (Neuron, 2011, 70, 1071-1084) and such methods are well known in the art.

The PCR amplification products are loaded onto gels, stained with ethidium bromide, and subjected to densitometry analysis. Mean intensities from regions of interest (ROI) that correspond to the bands of interest in the gel are measured.

Gene (or RNA) target quantities obtained by PCR are normalized using the expression level of a housekeeping gene whose expression is constant, such as GAPDH. Expression of the housekeeping gene (or RNA) is analyzed and measured using the same methods as the target.

Probes and primers are designed to hybridize to a C9ORF72 nucleic acid. Methods for designing RT-PCR probes and primers are well known in the art, and may include the use of software such as PRIMER EXPRESS Software (Applied Biosystems, Foster City, Calif.).

### Analysis of Protein Levels

Antisense inhibition of C9ORF72 nucleic acids can be assessed by measuring C9ORF72 protein levels. Protein levels of C9ORF72 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of mouse, rat, monkey, and human C9ORF72 are commercially available.

### In Vivo Testing of Antisense Compounds

Antisense compounds, for example, antisense oligonucleotides, are tested in animals to assess their ability to inhibit expression of C9ORF72 and produce phenotypic changes, such as, improved motor function and respiration. In certain embodiments, motor function is measured by rotarod, grip strength, pole climb, open field performance, balance beam, hindpaw footprint testing in the animal. In certain embodiments, respiration is measured by whole body plethysmograph, invasive resistance, and compliance measurements in the animal. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from CNS tissue or CSF and changes in C9ORF72 nucleic acid expression are measured.

### Targeting C9ORF72

Antisense oligonucleotides described herein may hybridize to a C9ORF72 nucleic acid derived from either DNA strand. For example, antisense oligonucleotides described herein may hybridize to a C9ORF72 antisense transcript or a C9ORF72 sense transcript. Antisense oligonucleotides described herein may hybridize to a C9ORF72 nucleic acid in any stage of RNA processing. Described herein are antisense oligonucleotides that are complementary to a pre-mRNA or a mature mRNA. Additionally, antisense oligonucleotides described herein may hybridize to any element of a C9ORF72 nucleic acid. For example, described herein are antisense oligonucleotides that are complementary to an exon, an intron, the 5′ UTR, the 3′ UTR, a repeat region, a hexanucleotide repeat expansion, a splice junction, an exon:exon splice junction, an exonic splicing silencer (ESS), an exonic splicing enhancer (ESE), exon 1a, exon 1b, exon 1c, exon 1d, exon 1e, exon 2, exon 3, exon 4, exon 5, exon 6, exon 7, exon 8, exon 9, exon 10, exon 11, intron 1, intron 2, intron 3, intron 4, intron 5, intron 6, intron 7, intron 8, intron 9, or intron 10 of a C9ORF72 nucleic acid.

In certain embodiments, antisense oligonucleotides described herein hybridize to all variants of C9ORF72 derived from the sense strand. In certain embodiments, the antisense oligonucleotides described herein selectively hybridize to certain variants of C9ORF72 derived from the sense strand. In certain embodiments, the antisense oligonucleotides described herein selectively hybridize to variants of C9ORF72 derived from the sense strand containing a hexanucleotide repeat expansion. In certain embodiments, the antisense oliognucleotides described herein selectively hybridize to pre-mRNA variants containing a hexanucleotide repeat. In certain embodiments, pre-mRNA variants of C9ORF72 containing a hexanucleotide repeat expansion include SEQ ID NO: 1-3 and 6-10. In certain embodiments, such hexanucleotide repeat expansion comprises at least 24 repeats of any of GGGGCC, GGGGGG, GGGGGC, or GGGGCG.

In certain embodiments, the antisense oligonucleotides described herein inhibit expression of all variants of C9ORF72 derived from the sense strand. In certain embodiments, the antisense oligonucleotides described herein inhibit expression of all variants of C9ORF72 derived from the sense strand equally. In certain embodiments, the antisense oligonucleotides described herein preferentially inhibit expression of one or more variants of C9ORF72 derived from the sense strand. In certain embodiments, the antisense oligonucleotides described herein preferentially inhibit expression of variants of C9ORF72 derived from the sense strand containing a hexanucleotide repeat expansion. In certain embodiments, the antisense oliognucleotides described herein selectively inhibit expression of pre-mRNA variants containing the hexanucleotide repeat. In certain embodiments, the antisense oliognucleotides described herein selectively inhibit expression of C9ORF72 pathogenic associated mRNA variants. In certain embodiments, pre-mRNA variants of C9ORF72 containing a hexanucleotide repeat expansion include SEQ ID NO: 1-3 and 6-10. In certain embodiments, such hexanucleotide repeat expansion comprises at least 24 repeats of any of GGGGCC, GGGGGG, GGGGGC, or GGGGCG. In certain embodiments, the hexanucleotide repeat expansion forms C9ORF72 sense foci. In certain embodiments, antisense oligonucleotides described herein are useful for reducing C9ORF72 sense foci. C9ORF72 sense foci may be reduced in terms of percent of cells with foci as well as number of foci per cell.

### C9OFF72 Features

Antisense oligonucleotides described herein may hybridize to any C9ORF72 nucleic acid at any state of processing within any element of the C9ORF72 gene. In certain embodiments, antisense oligonucleotides described herein may target the antisense transcript, e.g., SEQ ID NO: 11. In certain embodiments, antisense oligonucleotides described herein may hybridize to an exon, an intron, the 5′ UTR, the 3′ UTR, a repeat region, a hexanucleotide repeat expansion, a splice junction, an exon:exon splice junction, an exonic splicing silencer (ESS), an exonic splicing enhancer (ESE), exon 1a, exon 1b, exon 1c, exon 1d, exon 1e, exon 2, exon 3, exon 4, exon 5, exon 6, exon 7, exon 8, exon 9, exon 10, exon 11, intron 1, intron 2, intron 3, intron 4, intron 5, intron 6, intron 7, intron 8, intron 9, or intron 10. For example, antisense oligonucleotides may target any of the exons characterized below in Tables 1-5 described below. Antisense oligonucleotides described herein may also target nucleic acids not characterized below and such nucleic acid may be characterized in GENBANK. Moreover, antisense oligonucleotides described herein may also target elements other than exons and such elements as characterized in GENBANK.

TABLE 1
Functional Segments for NM_001256054.1 (SEQ ID NO: 1)

| | | | Start site | Stop site |
|---|---|---|---|---|
| | | | in | in |
| | mRNA | mRNA | reference | reference |
| Exon | start | stop | to SEQ | to SEQ |
| Number | site | site | ID NO: 2 | ID NO: 2 |
| exon 1C | 1 | 158 | 1137 | 1294 |
| exon 2 | 159 | 646 | 7839 | 8326 |
| exon 3 | 647 | 706 | 9413 | 9472 |
| exon 4 | 707 | 802 | 12527 | 12622 |
| exon 5 | 803 | 867 | 13354 | 13418 |
| exon 6 | 868 | 940 | 14704 | 14776 |
| exon 7 | 941 | 1057 | 16396 | 16512 |
| exon 8 | 1058 | 1293 | 18207 | 18442 |
| exon 9 | 1294 | 1351 | 24296 | 24353 |
| exon 10 | 1352 | 1461 | 26337 | 26446 |
| exon 11 | 1462 | 3339 | 26581 | 28458 |

TABLE 2
Functional Segments for NM_018325.3 (SEQ ID NO: 4)

| | | | Start site | Stop site |
|---|---|---|---|---|
| | | | in | in |
| | mRNA | mRNA | reference | reference |
| Exon | start | stop | to SEQ | to SEQ |
| Number | site | site | ID NO: 2 | ID NO: 2 |
| exon 1B | 1 | 63 | 1510 | 1572 |
| exon 2 | 64 | 551 | 7839 | 8326 |
| exon 3 | 552 | 611 | 9413 | 9472 |
| exon 4 | 612 | 707 | 12527 | 12622 |
| exon 5 | 708 | 772 | 13354 | 13418 |
| exon 6 | 773 | 845 | 14704 | 14776 |
| exon 7 | 846 | 962 | 16396 | 16512 |
| exon 8 | 963 | 1198 | 18207 | 18442 |
| exon 9 | 1199 | 1256 | 24296 | 24353 |
| exon 10 | 1257 | 1366 | 26337 | 26446 |
| exon 11 | 1367 | 3244 | 26581 | 28458 |

TABLE 3
Functional Segments for NM_145005.5 (SEQ ID NO: 6)

| | | | Start site | Stop site |
|---|---|---|---|---|
| | | | in | in |
| | mRNA | mRNA | reference | reference |
| Exon | start | stop | to SEQ | to SEQ |
| Number | site | site | ID NO: 2 | ID NO: 2 |
| exon 1A | 1 | 80 | 1137 | 1216 |
| exon 2 | 81 | 568 | 7839 | 8326 |
| exon 3 | 569 | 628 | 9413 | 9472 |
| exon 4 | 629 | 724 | 12527 | 12622 |
| exon 5B | 725 | 1871 | 13354 | 14500 |
| (exon 5 into | | | | |
| intron 5) | | | | |

TABLE 4
Functional Segments for DB079375.1 (SEQ ID NO: 7)

| | | | Start site | Stop site |
|---|---|---|---|---|
| | | | in | in |
| | mRNA | mRNA | reference | reference |
| Exon | start | stop | to SEQ | to SEQ |
| Number | site | site | ID NO: 2 | ID NO: 2 |
| exon 1E | 1 | 35 | 1135 | 1169 |
| exon 2 | 36 | 524 | 7839 | 8326 |
| exon 3 | 525 | 562 | 9413 | 9450 |
| (EST ends | | | | |
| before end | | | | |
| of full exon) | | | | |

TABLE 5
Functional Segments for BU194591.1 (SEQ ID NO: 8)

| | | | Start site | Stop site |
|---|---|---|---|---|
| | | | in | in |
| | mRNA | mRNA | reference | reference |
| Exon | start | stop | to SEQ | to SEQ |
| Number | site | site | ID NO: 2 | ID NO: 2 |
| exon 1D | 1 | 36 | 1241 | 1279 |
| exon 2 | 37 | 524 | 7839 | 8326 |
| exon 3 | 525 | 584 | 9413 | 9472 |
| exon 4 | 585 | 680 | 12527 | 12622 |
| exon 5B | 681 | 798 | 13354 | 13465 |
| (exon 5 into | | | | |
| intron 5) | | | | |

### Certain Indications

In certain embodiments, provided herein are methods of treating an individual comprising administering one or more pharmaceutical compositions described herein. In certain embodiments, the individual has a neurodegenerative disease. In certain embodiments, the individual is at risk for developing a neurodegenerative disease, including, but not limited to, ALS or FTD. In certain embodiments, the individual has been identified as having a C9ORF72 associated disease. In certain embodiments, the individual has been identified as having a C9ORF72 hexanucleotide repeat expansion associated disease. In certain embodiments, provided herein are methods for prophylactically reducing C9ORF72 expression in an individual. Certain embodiments include treating an individual in need thereof by administering to an individual a therapeutically effective amount of an antisense compound targeted to a C9ORF72 nucleic acid.

In one embodiment, administration of a therapeutically effective amount of an antisense compound targeted to a C9ORF72 nucleic acid is accompanied by monitoring of C9ORF72 levels in an individual, to determine an individual's response to administration of the antisense compound. An individual's response to administration of the antisense compound may be used by a physician to determine the amount and duration of therapeutic intervention.

In certain embodiments, administration of an antisense compound targeted to a C9ORF72 nucleic acid results in reduction of C9ORF72 expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a C9ORF72 nucleic acid results in improved motor function and respiration in an animal. In certain embodiments, administration of a C9ORF72 antisense compound improves motor function and respiration by at least 15, 20, 25, 30, 35, 40, 45, 50, 55, 60, 65, 70, 75, 80, 85, 90, 95 or 99%, or a range defined by any two of these values.

In certain embodiments, administration of an antisense compound targeted to a C9ORF72 antisense transcript results in reduction of C9ORF72 antisense transcript expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a C9ORF72 antisense transcript results in improved motor function and respiration in an animal. In certain embodiments, administration of a C9ORF72 antisense compound improves motor function and respiration by at least 15, 20, 25, 30, 35, 40, 45, 50, 55, 60, 65, 70, 75, 80, 85, 90, 95 or 99%, or a range defined by any two of these values. In certain embodiments, administration of a C9ORF72 antisense compound reduces the number of cells with C9ORF72 antisense foci and/or the number of C9ORF72 antisense foci per cell.

In certain embodiments, administration of an antisense compound targeted to a C9ORF72 sense transcript results in reduction of a C9ORF72 sense transcript expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a C9ORF72 sense transcript results in improved motor function and respiration in an animal. In certain embodiments, administration of a C9ORF72 antisense compound improves motor function and respiration by at least 15, 20, 25, 30, 35, 40, 45, 50, 55, 60, 65, 70, 75, 80, 85, 90, 95 or 99%, or a range defined by any two of these values. In certain embodiments, administration of a C9ORF72 antisense compound reduces the number of cells with C9ORF72 sense foci and/or the number of C9ORF72 sense foci per cell.

In certain embodiments, pharmaceutical compositions comprising an antisense compound targeted to a C9ORF72 nucleic are used for the preparation of a medicament for treating a patient suffering or susceptible to a neurodegenerative disease including ALS and FTD.

### Certain Combination Therapies

In certain embodiments, one or more pharmaceutical compositions described herein are co-administered with one or more other pharmaceutical agents. In certain embodiments, such one or more other pharmaceutical agents are designed to treat the same disease, disorder, or condition as the one or more pharmaceutical compositions described herein. In certain embodiments, such one or more other pharmaceutical agents are designed to treat a different disease, disorder, or condition as the one or more pharmaceutical compositions described herein. In certain embodiments, such one or more other pharmaceutical agents are designed to treat an undesired side effect of one or more pharmaceutical compositions described herein. In certain embodiments, one or more pharmaceutical compositions described herein are co-administered with another pharmaceutical agent to treat an undesired effect of that other pharmaceutical agent. In certain embodiments, one or more pharmaceutical compositions described herein are co-administered with another pharmaceutical agent to produce a combinational effect. In certain embodiments, one or more pharmaceutical compositions described herein are co-administered with another pharmaceutical agent to produce a synergistic effect.

In certain embodiments, one or more pharmaceutical compositions described herein and one or more other pharmaceutical agents are administered at the same time. In certain embodiments, one or more pharmaceutical compositions described herein and one or more other pharmaceutical agents are administered at different times. In certain embodiments, one or more pharmaceutical compositions described herein and one or more other pharmaceutical agents are prepared together in a single formulation. In certain embodiments, one or more pharmaceutical compositions described herein and one or more other pharmaceutical agents are prepared separately.

In certain embodiments, pharmaceutical agents that may be co-administered with a pharmaceutical composition described herein include Riluzole (Rilutek), Lioresal (Lioresal), and Dexpramipexole.

In certain embodiments, pharmaceutical agents that may be co-administered with a C9ORF72 antisense transcript specific inhibitor described herein include, but are not limited to, an additional C9ORF72 inhibitor. In certain embodiments, the co-adminstered pharmaceutical agent is administered prior to administration of a pharmaceutical composition described herein. In certain embodiments, the co-administered pharmaceutical agent is administered following administration of a pharmaceutical composition described herein. In certain embodiments the co-administered pharmaceutical agent is administered at the same time as a pharmaceutical composition described herein. In certain embodiments the dose of a co-administered pharmaceutical agent is the same as the dose that would be administered if the co-administered pharmaceutical agent was administered alone. In certain embodiments the dose of a co-administered pharmaceutical agent is lower than the dose that would be administered if the co-administered pharmaceutical agent was administered alone. In certain embodiments the dose of a co-administered pharmaceutical agent is greater than the dose that would be administered if the co-administered pharmaceutical agent was administered alone.

In certain embodiments, the co-administration of a second compound enhances the effect of a first compound, such that co-administration of the compounds results in an effect that is greater than the effect of administering the first compound alone. In other embodiments, the co-administration results in effects that are additive of the effects of the compounds when administered alone. In certain embodiments, the co-administration results in effects that are supra-additive of the effects of the compounds when administered alone. In certain embodiments, the first compound is an antisense compound. In certain embodiments, the second compound is an antisense compound.

### Certain Human Therapeutics

The human C9ORF72 antisense transcript specific antisense compounds described herein are being evaluated as possible human therapeutics. Various parameters of potency, efficacy, and/or tolerability are being examined. Such parameters include in vitro inhibition of C9ORF72 antisense transcript; in vitro dose response (IC50); in vivo inhibition of C9ORF72 antisense transcript in a transgenic animal containing a human C9ORF72 transgene in relevant tissues (e.g., brain and/or spinal cord); and/or tolerability in mouse, rat, dog, and/or primate. Tolerability markers that may be measured include blood and serum chemistry parameters, CSF chemistry parameters, body and organ weights, general observations and/or behavioral tests, and/or biochemical markers such as GFAP and/or AIF1. Acute or long term tolerability may be measured.

### Certain Assays for Measuring C90RF72 Antisense Transcripts

Certain assays described herein are directed to the reduction of C9ORF72 antisense transcript. Additional assays may be used to measure the reduction of C9ORF72 antisense transcript. Additional controls may be used as a baseline for measuring the reduction of C9ORF72 transcript.

### EXAMPLES

### Non-Limiting Disclosure and Incorporation by Reference

While certain compounds, compositions, and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of C9ORF72 Antisense Transcript

Antisense oligonucleotides targeted to C9ORF72 antisense transcript were tested for their effects on C9ORF72 antisense transcript expression in vitro. Cultured HepG2 cells were transfected with 50 nM antisense oligonucleotide or water for untransfected controls.

Total RNA was isolated from the cells 24 hours after transfection using TRIzol (Life Technologies) according to the manufacturer's directions. Two DNase reactions were performed, one on the column during RNA purification, and one after purification using amplification grade DNase. The isolated RNA was reverse transcribed to generate cDNA from the C9ORF72 antisense transcript using a primer of the sequence CGACTGGAGCACGAGGACACTGAGGAAAGAGAGGTGCGTCAAA (SEQ ID NO: 12). GAPDH cDNA was reverse transcribed as a control using a primer of the sequence TTTTTTTTTTTTTTTTTT (SEQ ID NO: 13).

Two PCR amplification steps were completed for the C9ORF72 antisense cDNA. The first PCR amplification was completed using outer forward primer AAAGAGAAGCAACCGGGC (SEQ ID NO: 14) and reverse primer CGACTGGAGCACGAGGACACTG (SEQ ID NO: 15). The PCR product of the first PCR amplification was subjected to a nested PCR using nested forward primer CAGGGACGGCTGACACA (SEQ ID NO: 16) and reverse primer SEQ ID 15. One PCR amplification of GAPDH was performed with forward primer GTCAACGGATTTGGTCGTATTG (SEQ ID NO: 17) and reverse primer TGGAAGATGGTGATGGGATTT (SEQ ID NO:18). The amplified cDNA was then loaded onto 5% acrylamide gels and stained with ethidium bromide. Densitometry analysis was performed using Gel Logic 200 and Kodak MI software (Kodak Scientific Imaging Systems, Rochester, N.Y., USA). The mean intensities from regions of interest (ROI) that corresponded to the C9ORF72 antisense cDNA and GAPDH cDNA bands were measured. The intensity of each C9ORF72 antisense cDNA band was normalized to its corresponding GAPDH cDNA band. These normalized values for the C9ORF72 antisense transcript expression for cells treated with antisense oligonucleotide were then compared to the normalized values for C9ORF72 antisense transcript expression in an untransfected control that was run in the same gel. The final value for band intensities obtained was used to calculate the % inhibition.

The table below lists the antisense oligonucleotides designed and tested for C9ORF72 antisense transcript expression in vitro. ISIS No. 141923 is a negative control that is mismatched to the target. Although ISIS No. 141923 is a negative control in that it is mismatched to the target, it does not necessarily represent a baseline for comparing C9ORF72 ASOs targeting the antisense transcript because it causes reduction of antisense transcript. ISIS No. 576816 is a negative control that is complementary to C9ORF72 sense transcript. ISIS No. 576816 causes no activity and represents a baseline for comparing the ASOs targeting the C9ORF72 antisense transcript. All of the oligonucleotides in the table are 5-10-5 gapmers, 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to a putative antisense transcript sequence (designated herein as SEQ ID NO: 11). SEQ ID NO: 11 is a sequence that is complementary to nucleotides 1159 to 1734 of SEQ ID NO: 2 (the complement of GENBANK Accession No. NT_008413.18 truncated from nucleotides 27535000 to 27565000).

TABLE 6
Inhibition of C9ORF72 Antisense Transcript by Antisense
Oligonucleotides

| ISIS | Start | Stop | | % | SEQ ID |
|---|---|---|---|---|---|
| NO. | Site | Site | Sequence 5′ to 3′ | Inhibition | NO |
| 141923 | n/a | n/a | CCTTCCCTGAAGGTTCCTCC | 27 | 19 |
| 576816 | n/a | n/a | GCCTTACTCTAGGACCAAGA | 0 | 20 |
| 664634 | 281 | 300 | GGAACTCAGGAGTCGCGCGC | 21 | 22 |
| 664633 | 284 | 303 | TCTGGAACTCAGGAGTCGCG | 14 | 23 |
| 664632 | 293 | 312 | GTAGCAAGCTCTGGAACTCA | 25 | 24 |
| 664631 | 296 | 315 | CCTGTAGCAAGCTCTGGAAC | 40 | 25 |
| 664630 | 299 | 318 | CAGCCTGTAGCAAGCTCTGG | 58 | 26 |
| 664628 | 305 | 324 | CAACCGCAGCCTGTAGCAAG | 51 | 28 |
| 664627 | 308 | 327 | AAACAACCGCAGCCTGTAGC | 24 | 29 |
| 664626 | 311 | 330 | GGGAAACAACCGCAGCCTGT | 35 | 30 |
| 664625 | 314 | 333 | GGAGGGAAACAACCGCAGCC | 22 | 31 |
| 664624 | 317 | 336 | CAAGGAGGGAAACAACCGCA | 14 | 32 |
| 664622 | 341 | 360 | TGATAAAGATTAACCAGAAG | 18 | 34 |
| 664621 | 344 | 363 | ACCTGATAAAGATTAACCAG | 2 | 35 |
| 664620 | 347 | 366 | AAGACCTGATAAAGATTAAC | 14 | 36 |
| 664619 | 353 | 372 | CAAGAAAAGACCTGATAAAG | 34 | 37 |
| 664618 | 356 | 375 | GAACAAGAAAAGACCTGATA | 4 | 38 |
| 664617 | 359 | 378 | GGTGAACAAGAAAAGACCTG | 16 | 39 |
| 664616 | 365 | 384 | GCTGAGGGTGAACAAGAAAA | 15 | 40 |
| 664615 | 368 | 387 | CTCGCTGAGGGTGAACAAGA | 37 | 41 |
| 664614 | 371 | 390 | GTACTCGCTGAGGGTGAACA | 31 | 42 |
| 664612 | 377 | 396 | CTCACAGTACTCGCTGAGGG | 11 | 44 |
| 664610 | 383 | 402 | CTTGCTCTCACAGTACTCGC | 79 | 46 |
| 664609 | 386 | 405 | CTACTTGCTCTCACAGTACT | 87 | 47 |
| 664608 | 389 | 408 | CCACTACTTGCTCTCACAGT | 73 | 48 |
| 664607 | 392 | 411 | TCCCCACTACTTGCTCTCAC | 66 | 49 |
| 664606 | 395 | 414 | CTCTCCCCACTACTTGCTCT | 91 | 50 |
| 664605 | 398 | 417 | CCTCTCTCCCCACTACTTGC | 50 | 51 |
| 664604 | 413 | 432 | TTTTGTTTTTCCCACCCTCT | 71 | 52 |
| 664603 | 428 | 447 | TTAGGAGGTGTGTGTTTTTG | 66 | 53 |
| 664602 | 431 | 450 | GGTTTAGGAGGTGTGTGTTT | 52 | 54 |
| 664601 | 434 | 453 | GTGGGTTTAGGAGGTGTGTG | 62 | 55 |
| 664598 | 443 | 462 | AGAGCAGGTGTGGGTTTAGG | 34 | 58 |
| 664597 | 446 | 465 | GCAAGAGCAGGTGTGGGTTT | 29 | 59 |
| 664595 | 452 | 471 | GGTCTAGCAAGAGCAGGTGT | 36 | 61 |

### Example 2

### In Vivo Rodent Inhibition and Tolerability with Treatment of C9ORF72 Antisense Oligonucleotides

In order to assess the tolerability of inhibition of C9ORF72 expression in vivo, antisense oligonucleotides targeting a murine C9ORF72 nucleic acid were designed and assessed in mouse and rat models.

ISIS 571883 was designed as a 5-10-5 MOE gapmer, 20 nucleosides in length, wherein the central gap segment comprises ten 2′-deoxynucleosides and is flanked by wing segments on both the 5′ end and on the 3′ end comprising five nucleosides each. Each nucleoside in the 5′ wing segment and each nucleoside in the 3′ wing segment has a MOE modification. The internucleoside linkages are phosphorothioate linkages. All cytosine residues throughout the gapmer are 5-methylcytosines. ISIS 571883 has a target start site of nucleoside 33704 on the murine C9ORF72 genomic sequence, designated herein as SEQ ID NO: 93 (the complement of GENBANK Accession No. NT_166289.1 truncated from nucleosides 3587000 to 3625000).

ISIS 603538 was designed as a 5-10-5 MOE gapmer, 20 nucleosides in length, wherein the central gap segment comprises ten 2′-deoxynucleosides and is flanked by wing segments on both the 5′ end and on the 3′ end comprising five nucleosides each. Each nucleoside in the 5′ wing segment and each nucleoside in the 3′ wing segment has a MOE modification. The internucleoside linkages are either phosphorothioate linkages or phosphate ester linkages (Gs Ao Co Co Gs Cs Ts Ts Gs As Gs Ts Ts Ts Gs Co Co Ao Cs A; wherein ‘s’ denotes a phosphorothioate internucleoside linkage, ‘o’ denotes a phosphate ester linkage; and A, G, C, T denote the relevant nucleosides). All cytosine residues throughout the gapmer are 5-methylcytosines. ISIS 603538 has a target start site of nucleoside 2872 on the rat C9ORF72 mRNA sequence, designated herein as SEQ ID NO: 94 (GENBANK Accession No. NM_001007702.1).

### Mouse Experiment 1

Groups of 4 C57BL/6 mice each were injected with 50 μg, 100 μg, 300 μg, 500 μg, or 700 μg of ISIS 571883 administered via an intracerebroventricular bolus injection. A control group of four C57/BL6 mice were similarly treated with PBS. Animals were anesthetized with 3% isofluorane and placed in a stereotactic frame. After sterilizing the surgical site, each mouse was injected −0.2 mm anterio-posterior from the bregma and 3 mm dorsoventral to the bregma with the above-mentioned doses of ISIS 571883 using a Hamilton syringe. The incision was closed with sutures. The mice were allowed to recover for 14 days, after which animals were euthanized according to a humane protocol approved by the Institutional Animal Care and Use Committee. Brain and spinal cord tissue were harvested and snap frozen in liquid nitrogen. Prior to freezing, brain tissue was cut transversely five sections using a mouse brain matrix.

### RNA Analysis

RNA was extracted from a 2-3 mm brain section posterior to the injection site, from brain frontal cortex and from the lumbar section of the spinal cord tissue for analysis of C9ORF72 mRNA expression. C9ORF72 mRNA expression was measured by RT-PCR. The data is presented in Table 7. The results indicate that treatment with increasing doses of ISIS 571883 resulted in dose-dependent inhibition of C9ORF72 mRNA expression.

The induction of the microglial marker AIF-1 as a measure of CNS toxicity was also assessed. The data is presented in Table 8. The results indicate that treatment with increasing doses of ISIS 571883 did not result in significant increases in AIF-1 mRNA expression. Hence, the injection of ISIS 571883 was deemed tolerable in this model.

TABLE 7
Percentage inhibition of C9ORF72 mRNA expression
compared to the PBS control

| | Posterior | | Spinal |
|---|---|---|---|
| Dose (μg) | brain | Cortex | cord |
| 50 | 22 | 8 | 46 |
| 100 | 22 | 12 | 47 |
| 300 | 55 | 47 | 67 |
| 500 | 61 | 56 | 78 |
| 700 | 65 | 65 | 79 |

TABLE 8
Percentage expression of AIF-1 mRNA expression
compared to the PBS control

| Dose | Posterior | Spinal |
|---|---|---|
| (μg) | brain | cord |
| 50 | 102 | 89 |
| 100 | 105 | 111 |
| 300 | 107 | 98 |
| 500 | 131 | 124 |
| 700 | 122 | 116 |

### Mouse Experiment 2

Groups of 4 C57BL/6 mice each were injected with 500 μg of ISIS 571883 administered via an intracerebroventricular bolus injection in a procedure similar to that described above. A control group of four C57/BL6 mice were similarly treated with PBS. The mice were tested at regular time points after ICV administration.

### Behavior Analysis

Two standard assays to assess motor behavior were employed; the rotarod assay and grip strength assay. In case of the rotarod assays, the time of latency to fall was measured. The data for the assays is presented in Tables 9 and 10. The results indicate that there were no significant changes in the motor behavior of the mice as a result of antisense inhibition of ISIS 571883 or due to the ICV injection. Hence, antisense inhibition of C9ORF72 was deemed tolerable in this model.

TABLE 9
Latency to fall (sec) in the rotarod assay

| Weeks after | | ISIS |
|---|---|---|
| injection | PBS | 571883 |
| 0 | 66 | 66 |
| 4 | 91 | 70 |
| 8 | 94 | 84 |

TABLE 10
Mean hindlimb grip strength (g) in the grip strength assay

| Weeks after | | ISIS |
|---|---|---|
| injection | PBS | 571883 |
| 0 | 57 | 63 |
| 1 | 65 | 51 |
| 2 | 51 | 52 |
| 3 | 51 | 51 |
| 4 | 59 | 72 |
| 5 | 60 | 64 |
| 6 | 61 | 72 |
| 7 | 67 | 68 |
| 8 | 66 | 70 |
| 9 | 63 | 61 |
| 10 | 48 | 46 |

### Rat Experiment

Groups of 4 Sprague-Dawley rats each were injected with 700 μg, 1,000 μg, or 3,000 μg of ISIS 603538 administered via an intrathecal bolus injection. A control group of four Sprague-Dawley rats was similarly treated with PBS. Animals were anesthetized with 3% isofluorane and placed in a stereotactic frame. After sterilizing the surgical site, each rat was injected with 30 μL of ASO solution administered via 8 cm intrathecal catheter 2 cm into the spinal canal with a 50 μL flush. The rats were allowed to recover for 4 weeks, after which animals were euthanized according to a humane protocol approved by the Institutional Animal Care and Use Committee.

### RNA Analysis

RNA was extracted from a 2-3 mm brain section posterior to the injection site, from brain frontal cortex, and from the cervical and lumbar sections of the spinal cord tissue for analysis of C9ORF72 mRNA expression. C9ORF72 mRNA expression was measured by RT-PCR. The data is presented in Table 11. The results indicate that treatment with increasing doses of ISIS 603538 resulted in dose-dependent inhibition of C9ORF72 mRNA expression.

The induction of the microglial marker AIF-1 as a measure of CNS toxicity was also assessed. The data is presented in Table 12. The results indicate that treatment with increasing doses of ISIS 603538 did not result in significant increases in AIF-1 mRNA expression. Hence, the injection of ISIS 603538 was deemed tolerable in this model.

TABLE 11
Percentage inhibition of C9ORF72 mRNA expression
compared to the PBS control

| Dose | Brain (1 mm | | Spinal cord | Spinal cord |
|---|---|---|---|---|
| (μg) | section) | Cortex | (lumbar) | (cervical) |
| 700 | 21 | 4 | 86 | 74 |
| 1000 | 53 | 49 | 88 | 82 |
| 3000 | 64 | 62 | 88 | 80 |

TABLE 12
Percentage expression of AIF-1 mRNA expression
compared to the PBS control

| Dose | Brain (1 mm | | Spinal cord | Spinal cord |
|---|---|---|---|---|
| (μg) | section) | Cortex | (lumbar) | (cervical) |
| 700 | 97 | 119 | 98 | 89 |
| 1000 | 105 | 113 | 122 | 96 |
| 3000 | 109 | 141 | 156 | 115 |

### Body Weight Analysis

Body weights of the rats were measured at regular time point intervals. The data is presented in Table 13. The results indicate that treatment with increasing doses of ISIS 603538 did not have any significant changes in the body weights of the rats.

TABLE 13
Body weights of the rats (% initial body weight)

| | Dose | | | | | |
|---|---|---|---|---|---|---|
| | (μg) | Week 1 | Week 2 | Week 3 | Week 4 | Week 5 |
| PBS | | 100 | 94 | 103 | 105 | 109 |
| ISIS | 700 | 100 | 94 | 98 | 103 | 107 |
| 603538 | 1000 | 100 | 95 | 97 | 101 | 103 |
| | 3000 | 100 | 92 | 98 | 102 | 105 |

### Example 3

### Dose Response Screens of Antisense Oligonucleotides Targeting Human C9ORF72 Sense Transcript in Two Patient Fibroblast Lines

Two different fibroblast cell lines from human patients (F09-152 and F09-229) were analyzed with antisense oligonucleotides that target the C9ORF72 sense transcript before exon 1B; i.e. antisense oligonucleotides that target the hexanucleotide repeat expansion containing transcript and antisense oligonucleotides that target downstream of exon 1. The target start and stop sites and the target regions with respect to SEQ ID NOs: 1 and 2 for each oligonucleotide are provided in Table 14. ISIS 577061 and ISIS 577065 target C9ORF72 upstream of exon 1B and just upstream of the hexanucleotide repeat. The rest of the ISIS oligonucleotides of Table 15 target C9ORF72 downstream of exon 1B and the hexanucleotide repeat.

TABLE 14
Target Start and Stop sites of ISIS oligonucleotides used in a
dose response assay in C9ORF72 patient fibroblasts

| | Target | Target | |
|---|---|---|---|
| | Start Site | Start Site | |
| ISIS | at SEQ ID | at SEQ ID | Target |
| No | NO: 1 | NO: 2 | Region |
| 577061 | n/a | 1406 | Upstream of |
| | | | exon 1B |
| 577065 | n/a | 1446 | Upstream of |
| | | | exon 1B |
| 577083 | n/a | 3452 | Downstream |
| | | | of exon 1B |
| 576816 | 232 | 7990 | Exon 2 |
| 576974 | 3132 | 28251 | Exon 11 |

Cells were plated at a density of 20,000 cells per well and transfected using electroporation with 246.9 nM, 740.7 nM, 2,222.2 nM, 6,666.7 nM, and 20,000.0 nM concentrations of antisense oligonucleotide. After a treatment period of approximately 16 hours, RNA was isolated from the cells and C9ORF72 mRNA levels were measured by quantitative real-time PCR. Two primer probe sets were used: (1) human C9ORF72 primer probe set RTS3750, which measures total mRNA levels, and (2) RTS3905, which targets the hexanucleotide repeat expansion containing transcript, which measures only mRNA transcripts that contain the hexanucleotide repeat expansion. C9ORF72 mRNA levels were adjusted according to total RNA content, as measured by RIBOGREEN®. Results are presented as percent inhibition of C9ORF72, relative to untreated control cells.

As illustrated in Tables 15-18, below, the two oligonucleotides that target upstream of exon 1B and, therefore, target mRNA transcripts containing the hexanucleotide repeat expansion (ISIS 577061 and ISIS 577065), do not inhibit total mRNA levels of C9ORF72 (as measured by RTS3750) as well as ISIS 576974, 576816, and 577083, which target downstream of exon 1B and, therefore, do not target the mRNA transcript containing the hexanucleotide repeat expansion. Expression levels of the C9ORF72 mRNA transcript containing the hexanucleotide repeat expansion are low (about 10% of the total C9ORF72 expression products), therefore, oligonucleotides targeting the mRNA transcript containing the hexanucleotide repeat expansion do not robustly inhibit total C9ORF72 mRNA (as measured by RTS3905), as suggested by Tables 16 and 18 below. Thus, ISIS 577061 and ISIS 577065 preferentially inhibit expression of mRNA transcripts containing the hexanucleotide repeat expansion.

TABLE 15
Percent inhibition of C9ORF72 total mRNA in F09-152 patient
fibroblasts in a dose response assay as measured with RTS3750

| | | | | | 20000.0 |
|---|---|---|---|---|---|
| ISIS No | 246.9 nM | 740.7 nM | 2222.2 nM | 6666.7 nM | nM |
| 577061 | 6 | 11 | 0 | 18 | 10 |
| 577065 | 10 | 11 | 30 | 29 | 0 |
| 576974 | 61 | 69 | 72 | 83 | 83 |
| 576816 | 35 | 76 | 82 | 91 | 93 |
| 577083 | 28 | 38 | 52 | 75 | 80 |

TABLE 16
Percent inhibition of C9ORF72 mRNA transcripts containing the
hexanucleotide repeat expansion in F09-152 patient fibroblasts
in a dose response assay as measured with RTS3905

| | | | | | 20000.0 |
|---|---|---|---|---|---|
| ISIS No | 246.9 nM | 740.7 nM | 2222.2 nM | 6666.7 nM | nM |
| 577061 | 4 | 28 | 58 | 81 | 87 |
| 577065 | 25 | 54 | 70 | 90 | 94 |
| 576974 | 57 | 77 | 81 | 93 | 92 |
| 576816 | 37 | 77 | 91 | 97 | 98 |
| 577083 | 37 | 53 | 74 | 93 | 94 |

TABLE 17
Percent inhibition of C9ORF72 total mRNA in F09-229 patient
fibroblasts in a dose response assay as measured with RTS3750

| | | | | | 20000.0 |
|---|---|---|---|---|---|
| ISIS No | 246.9 nM | 740.7 nM | 2222.2 nM | 6666.7 nM | nM |
| 577061 | 0 | 0 | 0 | 17 | 7 |
| 577065 | 8 | 17 | 17 | 16 | 3 |
| 576974 | 43 | 58 | 85 | 85 | 74 |
| 576816 | 45 | 70 | 85 | 81 | 89 |
| 577083 | 22 | 45 | 56 | 76 | 78 |

TABLE 18
Percent inhibition of C9ORF72 mRNA transcripts containing the
hexanucleotide repeat expansion in F09-229 patient fibroblasts
in a dose response assay as measured with RTS3905

| | | | | | 20000.0 |
|---|---|---|---|---|---|
| ISIS No | 246.9 nM | 740.7 nM | 2222.2 nM | 6666.7 nM | nM |
| 577061 | 14 | 36 | 70 | 87 | 89 |
| 577065 | 26 | 48 | 92 | 91 | 98 |
| 576974 | 63 | 87 | 91 | 92 | 91 |
| 576816 | 62 | 81 | 96 | 98 | 100 |
| 577083 | 36 | 64 | 82 | 98 | 96 |

What is claimed is:

1. A compound comprising a C9ORF72 antisense transcript specific inhibitor.

2. The compound of claim 1 wherein the C9ORF72 antisense transcript specific inhibitor is a C9ORF72 antisense transcript specific antisense compound.

3. The compound of claim 2, wherein the C9ORF72 antisense transcript specific antisense compound comprises an antisense oligonucleotide.

4. The compound of claim 3, wherein the antisense oligonucleotide consists of 12-30 linked nucleosides.

5. The compound of claim 3, wherein the antisense oligonucleotide consists of 16-25 linked nucleosides.

6. The compound of claim 3, wherein the antisense oligonucleotide consists of 18-22 linked nucleosides

7. The compound of any of claims 3-6, wherein the antisense oligonucleotide has a nucleobase sequence that is at least 80%, at least 85%, at least 90%, at least 91%, at least 92%, at least 93%, at least 94%, at least 95%, at least 96%, at least 97%, at least 98%, at least 99%, or 100% complementary to a C9ORF72 antisense transcript.

8. The compound of any of claims 3-6, wherein the antisense oligonucleotide has a nucleobase sequence that is at least 90% complementary to a C9ORF72 antisense transcript.

9. The compound of any of claims 3-6, wherein the antisense oligonucleotide has a nucleobase sequence that is at least 95% complementary to a C9ORF72 antisense transcript.

10. The compound of any of claims 3-6, wherein the antisense oligonucleotide has a nucleobase sequence that is 100% complementary to a C9ORF72 antisense transcript.

11. The compound of any of claims 10-13, wherein the C9ORF72 antisense transcript is SEQ ID NO: 11.

12. The compound of any of claims 3-10, wherein the antisense oligonucleotide has a nucleobase sequence comprising least 8, at least 9, at least 10, at least 11, at least 12, at least 13, at least 14, at least 15, at least 16, at least 17, at least 18, at least 19, or 20 contiguous nucleobases of a sequence selected from among SEQ ID NO: 19-20, 22-26, 28-32, 34-42, 44, 46-55, 58-59, and 61.

13. The compound of claim 3-11, wherein the antisense oligonucleotide is a modified antisense oligonucleotide.

14. The compound of claim 12, wherein the modified antisense oligonucleotide comprises at least one modified internucleoside linkage.

15. The compound of claim 13, wherein at least one modified internucleoside linkage is a phosphorothioate internucleoside linkage.

16. The compound of claim 14, wherein each modified internucleoside linkage is a phosphorothioate internucleoside linkage.

17. The compound of any of claims 12-15 wherein the modified antisense oligonucleotide comprises at least one phosphodiester internucleoside linkage.

18. The compound of claims 12-15, wherein at least one nucleoside of the modified antisense oligonucleotide comprises a modified nucleobase.

19. The compound of claim 16, wherein the modified nucleobase is a 5-methylcytosine.

20. The compound of claims 12-17, wherein at least one nucleoside of the modified antisense oligonucleotide comprises a modified sugar.

21. The compound of claim 18, wherein the at least one modified sugar is a bicyclic sugar.

22. The compound of claim 19, wherein the bicyclic sugar comprises a chemical bridge between the 2′ and 4′ position of the sugar, wherein the chemical bridge is selected from: 4′-CH₂—O-2′; 4′-CH(CH₃)—O-2′; 4′-(CH₂)₂—O-2′; and 4′-CH₂—N(R)—O-2′ wherein R is, independently, H, C₁-C₁₂ alkyl, or a protecting group.

23. The compound of claim 18, wherein at least one modified sugar comprises a 2′-O-methoxyethyl group.

24. The compound of any of claims 12-22, wherein the antisense oligonucleotide is a gapmer.

25. The compound of any of claims 12-23, wherein the antisense oligonucleotide is single-stranded.

26. The compound of any of claims 2-24, wherein the C9ORF72 antisense transcript specific antisense compound comprises at least one conjugate.

27. The compound of any of claims 2-24, wherein the C9ORF72 antisense transcript specific antisense compound consists of an antisense oligonucleotide.

28. A pharmaceutical composition comprising the compound of any of claims 1-26 and a pharmaceutically acceptable diluent or carrier.

29. A pharmaceutical composition of claim 27 further comprising a C9ORF72 sense transcript specific inhibitior.

30. The pharmaceutical composition of claim 28, wherein the C9ORF72 sense transcript specific inhibitor is a C9ORF72 sense transcript specific antisense compound.

31. The pharmaceutical composition of claim 29, wherein the C9ORF72 sense transcript specific antisense compound is an antisense oligonucleotide.

32. A pharmaceutical composition comprising a C9ORF72 antisense transcript specific inhibitor and a C9ORF sense transcript specific inhibitior.

33. The pharmaceutical composition of claim 32, wherein the C9ORF72 sense transcript specific inhibitor is a C9ORF72 sense transcript specific antisense compound.

34. The pharmaceutical composition of claim 32, wherein the C9ORF72 antisense transcript specific inhibitor is a C9ORF72 antisense transcript specific antisense compound.

35. The pharmaceutical composition of claim 33, wherein the C9ORF72 sense transcript specific antisense compound is an antisense oligonucleotide

36. The pharmaceutical composition of claim 34, wherein the C9ORF72 antisense transcript specific antisense compound is an antisense oligonucleotide.

37. The pharmaceutical composition of claims 29-36, wherein the antisense transcript is SEQ ID NO: 11.

38. The pharmaceutical composition of claims 29-36, wherein the antisense transcript is any of SEQ ID NOs: 1-10.

39. Use of the compound of any preceding claim for the manufacture of a medicament for treating a neurodegenerative disease.


SEQUENCE LISTING (94 sequences)

SEQ ID NO: 1 - DNA, 3339 bp, Homo sapiens
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttg

SEQ ID NO: 2 - DNA, 30001 bp, Homo sapiens
caaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctatgcaaggttcattttgtaaatattggaaatggagtgataatacgtacttcaccagaggatttaatgagaccttatacgatccttagttcagtacctgactagtgcttcataaatgctttttcatccaatctgacaatctccagcttgtaattggggcatttagaacatttaatatgattattggcatggtaggttaaagctgtcatcttgctgttttctatttgttctttttgttttctccttacttttggatttttttattctactatgtcttttctattgtcttattaactatactctttgatttattttagtggttgttttagggttatacctctttctaatttaccagtttataaccagtttatatactacttgacatatagcttaagaaacttactgttgttgtctttttgctgttatggtcttaacgtttttatttctacaaacattataaactccacactttattgttttttaattttacttatacagtcaattatcttttaaagatatttaaatataaacattcaaaacaccccaat

SEQ ID NO: 3 - DNA, 1031 bp, Homo sapiens
attcccgggatacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaaatgtccagaagattatcttagaaggcacagagagaatggaagatcagggtcagagtattattccaatgcttactggagaagtgattcctgtaatggaaactgctttcctctatgaaattcccccgggttcctggaggaaatagatataggctgatacagttacccaatgatggatgaatattgggggaccgcctggtcattgaaaggctttcttttctccaggaaagaaatttttttccttttccataaaaagcttgggaatggaagacaacaattcccattctttttttgcgttccacccctatgtgacaacagaaatttttggggaaacaacaacgaaaaaattttatcccgcgcgca

SEQ ID NO: 4 - DNA, 3244 bp, Homo sapiens
gggcggggctgcggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttg

SEQ ID NO: 5 - DNA, 761 bp, Homo sapiens
  misc_feature (693)..(693): n is a, c, g, or t
  misc_feature (722)..(722): n is a, c, g, or t
cacgaggctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcangatacgatcatctacactgacgaaagctntactcctgatttgaatatttttcaagatgtcttacacag

SEQ ID NO: 6 - DNA, 1901 bp, Homo sapiens
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggcaaaaaaaaaaaaaaaaaaaaaaaaaaaa

SEQ ID NO: 7 - DNA, 562 bp, Homo sapiens
  misc_feature (166)..(166): n is a, c, g, or t
agacgtaacctacggtgtcccgctaggaaagagagatatctccggagcatttggataatgtgacagttggaatgcagtgatgtcgactctttgcccaccgccatctccagctgttgccaagacagagattgctttaagtggcaaatcacctttattagcagctacnttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaagg

SEQ ID NO: 8 - DNA, 798 bp, Homo sapiens
gggctctcttttgggggcggggtctagcaagagcagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatgggactgctttcatctatgaaatcacacagtgttcctgaagaaatagatatagctgatacagtactccatgatgatgatatttggtgacagctgtcatgaaaggctttcttctcaagtaggaattttttcttttcataaaagctgggatgaagccagattcccatct

SEQ ID NO: 9 - DNA, 169 bp, Homo sapiens
aaacagcgacaagttccgcccacgtaaaagatgatgcttggtgtgtcagccgtccctgctgcccggttgcttctcttttgggggcggggtctagcaagagcagatatctccggagcatttggataatgtgacagttggaatgcggtgatgtcgactctttgcccaccgc

SEQ ID NO: 10 - DNA, 176 bp, Homo sapiens
aaaacgtcatcgcacatagaaaacagacagacgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgc

SEQ ID NO: 11 - DNA, 576 bp, Homo sapiens
agtcgctagaggcgaaagcccgacacccagcttcggtcagagaaatgagagggaaagtaaaaatgcgtcgagctctgaggagagcccccgcttctacccgcgcctcttcccggcagccgaaccccaaacagccacccgccaggatgccgcctcctcactcacccactcgccaccgcctgcgcctccgccgccgcgggcgcaggcaccgcaaccgcagccccgccccgggcccgcccccgggcccgccccgaccacgccccggccccggccccggcccctagcgcgcgactcctgagttccagagcttgctacaggctgcggttgtttccctccttgttttcttctggttaatctttatcaggtcttttcttgttcaccctcagcgagtactgtgagagcaagtagtggggagagagggtgggaaaaacaaaaacacacacctcctaaacccacacctgctcttgctagaccccgcccccaaaagagaagcaaccgggcagcagggacggctgacacaccaagcgtcatcttttacgtgggcggaacttgtcgctgtttgacgcacctctctttcct

SEQ ID NO: 12 - DNA, 43 bp, Artificial sequence
  Primer
cgactggagcacgaggacactgaggaaagagaggtgcgtcaaa

SEQ ID NO: 13 - DNA, 18 bp, Artificial sequence
  Primer
tttttttttttttttttt

SEQ ID NO: 14 - DNA, 18 bp, Artificial sequence
  Primer
aaagagaagcaaccgggc

SEQ ID NO: 15 - DNA, 22 bp, Artificial sequence
  Primer
cgactggagcacgaggacactg

SEQ ID NO: 16 - DNA, 17 bp, Artificial sequence
  Primer
cagggacggctgacaca

SEQ ID NO: 17 - DNA, 22 bp, Artificial sequence
  Primer
gtcaacggatttggtcgtattg

SEQ ID NO: 18 - DNA, 21 bp, Artificial sequence
  Primer
tggaagatggtgatgggattt

SEQ ID NO: 19 - DNA, 20 bp, Artificial sequence
  Synthetic oligonucleotide
ccttccctgaaggttcctcc

SEQ ID NO: 20 - DNA, 20 bp, Artificial sequence
  Synthetic oligonucleotide
gccttactctaggaccaaga

SEQ ID NO: 21 - skipped

SEQ ID NO: 22 - DNA, 20 bp, Artificial sequence
  Synthetic oligonucleotide
ggaactcaggagtcgcgcgc

SEQ ID NO: 23 - DNA, 20 bp, Artificial sequence
  Synthetic oligonucleotide
tctggaactcaggagtcgcg

SEQ ID NO: 24 - DNA, 20 bp, Artificial sequence
  Synthetic oligonucleotide
gtagcaagctctggaactca

SEQ ID NO: 25 - DNA, 20 bp, Artificial sequence
  Synthetic oligonucleotide
cctgtagcaagctctggaac

SEQ ID NO: 26 - DNA, 20 bp, Artificial sequence
  Synthetic oligonucleotide
cagcctgtagcaagctctgg

SEQ ID NO: 27 - skipped

SEQ ID NO: 28 - DNA, 20 bp, Artificial sequence
  Synthetic oligonucleotide
caaccgcagcctgtagcaag

SEQ ID NO: 29 - DNA, 20 bp, Artificial sequence
  Synthetic oligonucleotide
aaacaaccgcagcctgtagc

SEQ ID NO: 30 - DNA, 20 bp, Artificial sequence
  Synthetic oligonucleotide
gggaaacaaccgcagcctgt

SEQ ID NO: 31 - DNA, 20 bp, Artificial sequence
  Synthetic oligonucleotide
ggagggaaacaaccgcagcc

SEQ ID NO: 32 - DNA, 20 bp, Artificial sequence
  Synthetic oligonucleotide
caaggagggaaacaaccgca

SEQ ID NO: 33 - skipped

SEQ ID NO: 34 - DNA, 20 bp, Artificial sequence
  Synthetic oligonucleotide
tgataaagattaaccagaag

SEQ ID NO: 35 - DNA, 20 bp, Artificial sequence
  Synthetic oligonucleotide
acctgataaagattaaccag

SEQ ID NO: 36 - DNA, 20 bp, Artificial sequence
  Synthetic oligonucleotide
aagacctgataaagattaac

SEQ ID NO: 37 - DNA, 20 bp, Artificial sequence
  Synthetic oligonucleotide
caagaaaagacctgataaag

SEQ ID NO: 38 - DNA, 20 bp, Artificial sequence
  Synthetic oligonucleotide
gaacaagaaaagacctgata

SEQ ID NO: 39 - DNA, 20 bp, Artificial sequence
  Synthetic oligonucleotide
ggtgaacaagaaaagacctg

SEQ ID NO: 40 - DNA, 20 bp, Artificial sequence
  Synthetic oligonucleotide
gctgagggtgaacaagaaaa

SEQ ID NO: 41 - DNA, 20 bp, Artificial sequence
  Synthetic oligonucleotide
ctcgctgagggtgaacaaga

SEQ ID NO: 42 - DNA, 20 bp, Artificial sequence
  Synthetic oligonucleotide
gtactcgctgagggtgaaca

SEQ ID NO: 43 - skipped

SEQ ID NO: 44 - DNA, 20 bp, Artificial sequence
  Synthetic oligonucleotide
ctcacagtactcgctgaggg

SEQ ID NO: 45 - skipped

SEQ ID NO: 46 - DNA, 20 bp, Artificial sequence
  Synthetic oligonucleotide
cttgctctcacagtactcgc

SEQ ID NO: 47 - DNA, 20 bp, Artificial sequence
  Synthetic oligonucleotide
ctacttgctctcacagtact

SEQ ID NO: 48 - DNA, 20 bp, Artificial sequence
  Synthetic oligonucleotide
ccactacttgctctcacagt

SEQ ID NO: 49 - DNA, 20 bp, Artificial sequence
  Synthetic oligonucleotide
tccccactacttgctctcac

SEQ ID NO: 50 - DNA, 20 bp, Artificial sequence
  Synthetic oligonucleotide
ctctccccactacttgctct

SEQ ID NO: 51 - DNA, 20 bp, Artificial sequence
  Synthetic oligonucleotide
cctctctccccactacttgc

SEQ ID NO: 52 - DNA, 20 bp, Artificial sequence
  Synthetic oligonucleotide
ttttgtttttcccaccctct

SEQ ID NO: 53 - DNA, 20 bp, Artificial sequence
  Synthetic oligonucleotide
ttaggaggtgtgtgtttttg

SEQ ID NO: 54 - DNA, 20 bp, Artificial sequence
  Synthetic oligonucleotide
ggtttaggaggtgtgtgttt

SEQ ID NO: 55 - DNA, 20 bp, Artificial sequence
  Synthetic oligonucleotide
gtgggtttaggaggtgtgtg

SEQ ID NO: 56-57 - skipped (2 entries)

SEQ ID NO: 58 - DNA, 20 bp, Artificial sequence
  Synthetic oligonucleotide
agagcaggtgtgggtttagg

SEQ ID NO: 59 - DNA, 20 bp, Artificial sequence
  Synthetic oligonucleotide
gcaagagcaggtgtgggttt

SEQ ID NO: 60 - skipped

SEQ ID NO: 61 - DNA, 20 bp, Artificial sequence
  Synthetic oligonucleotide
ggtctagcaagagcaggtgt

SEQ ID NO: 62-92 - skipped (31 entries)

SEQ ID NO: 93 - DNA, 38001 bp, Mus musculus
tgtctctaggtaaaattttgaaggaaaaaaaaaacactaagaaggtatattccttcaaagttccagtcttattctgaagtgtaatgttatgttagtttgactcacagacaggttttaaagaagggcttacttcaagaggacaccaaacaaataccttctattcctagtgggctctggaatcacagaaaactgacccaatcaattacattgatagctctggcttactacagacaagcaaattatcttaagtgtgcatgcatgcgcgtgtatgtgtgttagtacctaacacccacctgggaacttttcagcttttcagtgtgggatatagtataaacgtctattcctcgtgttgtggattagctgactggcctcactcagctgccttccttacctgcaaactcacccactttgactacagcatcgcactcttaaccctagccttccaaacatggtcctatgctatttctgtgtgtctggatgtatttttaactctcagatgtatacttcatttatgagatatacatctgaagaccacggtacaaaacactgtaagaacttgatagaatgacaactgctaggtaaaaaaaaaaaaaaaaaaaaaaaaaaaaaaaaaaaaaagcatacaatacctggtgagagttctatttttaccgaaggtggtattgataggtattctgttattaatgcctttcttttccctataaatgatgaaaagttgctggaaaataataaacactactcatctgtagtgaaaagccacaatacagttacaaaccaatcaatcaatcaataaatcagacgtcatggtgttcttttcccaaaggttaaaaaacaaagtgcactgtgctatttggcaaaaatgacgtttagaagaaaacacggtgactacgcacagagggtgggggaatcattgtgcttgttgcggagtgaacacgtacagtgtgcacgcagacttacggcatttaaccgtgtcatagggaccaaaggaaatccactcactcactaaatatttgttgagcacccactacctgccaactcccaaacaaaacaaagcaaaactacttacaaccacaaactacgcttcgtaacctagatagataacgcaggtgacactatctatctaggttgagctcagctctgcccatgcttttcctgagcggctcttggaagaaaagctacaaagcccatgacagcctccgcctggccagctgccactggcatctcaaggctggcaaagcaaagtgaaagcgccaacccggaacttacggagtcccacgagggaaccgcggcgcgtcaagcagagacgagttccgcccacgtgaaagatggcgtttgtagtgacagccatcccaattgccctttccttctaggtggaaagtggtgtctagacagtccagggagggtgtgcgagggaggtgcgttttggttgcctcagctcgcaacttaactccacaacggtgaccaaggacaaaagaaggaaacaagactgcagagatccgcaccggggagccctgcagattctgggtctgctgtggactgggggcgggactgcgactgggcgggcctgggggcgtgtccggggcggggcggtcccggggcggggcccggagcgggctgcggttgcggtccctgcgccggcggtgaaggcgcagcagcggcgagtgggtgagtgagacgcgcgggcggaggggggctgctgccacggtcggctcgcgggccggccggctccgggtaccagcggggtttttttctccttcgaggtgaactcctccctgtcccccgggcgaaagagcccttggccttgcaggagttgcgggggccgcggcggtgcggaggggatggggatgggcctcatctttgctgtccgcccgcgctccccgatcccgacccggagcgtctcccgggcccttgagggaaccctccgggagtacggcgagcgcggcccccaccgccacaagcctgggccccaggggcctggcccggcgacagctggtgggtcctgcgacccagtcaggtctcccgagggtccccgcccgggaggagaaagcgccggtgggatggagtaaggacggacagaacaacacgcaggcaggatttcgcagaagtttgcaaggagtgcggatgcccacttacatgggctgctactcttaccaggttgttccccagttctgtgggacgtgacctggttgcctcacagctccgcggttgtacagacttattaaaggaagtgaccattgtgacttgggcatcacttgactgatggtaatcagttgcagagagagaagtgcactgattaagtctgtccacacagggtctgtctggccaggagtgcatttgcctgggagggattggttgcgctttctggtgtggggactattaggctcttgtagagttttgtcccggcagatggataaatttcttgttacactgttcccgttcgtcaccagttgagaaaaacgggtacacagtctgtctcagtagtacttttactttatattaagggcccaaaagggactggaaaatactttaagatagaatcgttagtccacttggaaaacttaaaatatgagagagagaggggggggggagagagagagagagagagagagagaaaggaaggaagaaggaggaagaggaggaggaaagagattgagattatgttaataatatggaatcagaatatttgaaatatagtaagcgtcccctcagttaaagaggacattccaggaggcccccagtatagcctgaaatctcaggaaacgcctacatacacccatcgtgtggatataggtgttttcccttcattacatttcatacacagatgttaaagtttagaaagtaggcacaataagagattacaaataactgataataaagtcgagccattgcagctgctctgtaaaagtcctgtgaatgtgatcgctttgtgtttcaaagtaacttactgtacttcacccctgttaagcaaaacaagattcacctgaacgcaggcaccttggtaccttggcagacaccagatctgataaccaagaggatggagaagtagtggcagacagtgtggagagcatgaatatgctagacaaaagggtgaatcataacctaggagcagaaagcaggtatttcatcatcctccacagtaaaaacctatgtcacgtaaaaaacctacaagtagtttttcttttactctttttgaatgaaagcttgctacaggcactgaaagttaaaataatctgtggatcaggaggaacaggggttttctgtctgagtcactgctgactagcacctcagtgaccattggcactgtgggaaaccccagagtcagttggaaacttcgaaactaaaggtgacggtgttcttatttcatagaacacaaaaaataagaggggttacagcctgcgctgcagactggacattcaacaagcatttaaatttctgggagacaaatgtaaatataactttaaaagttggtaaaatactctgtttggctatgttggccatccaatgtttgcttttagaaaatgactgaatggataaaacgtctatcttttgagcctgccctagacccccatgttgagtgaatactgtccaagtgttaggttagccggcctgagaaacttggatctaggcaagatggcacagtcctggtgtcatgagtatgcatgtgagttttggctgaaattgaacatttgtagagaatgacaaaggctggtctggcaagtagtccactgtctttacagtggtcttggttagttcctgtttggctgagagggctggttgatggctgtcctgcccctcttcccacaagtggaagccttatggtataattcttgatcacagtagcagtaggcaaatgaacttcctcaaagcagcctggaaagctgatttttttttctttctttctcttttttttttttttttcacaaggttaaagaaaaaacaaagggcttcaaatgtgccagtctgctaacagtgttaacatgtttattaacataaataaactttattagtttttggaagtattggttaagccctcgtgacccctgaactcggtttatagagtgatgagtcgtagcctcactctggtttggactctggcttctctcagaagactctgtggctaatgttaaccttctgaagtagccagaaaacatataagcaaaagtctgtgaggttgaaatgaattttttggccacatttgtatatgggttcccaccaatgctaacttcaggtgttagtaatatcagactcacagcttccctgattacacttcgctataagactttattttttaggtcataggaatttcccctttttcatgattcctaaatcatgaaataacatagtctaaaaatacggtattcctgaaataaacaatttctaagttttaagctgcgtgctattctgaacagtctgatgccctcttgtagcttttactgtgtcctaccccgggcatggttgattcctttgtccaaacatctgtctgttgtatccacactggattgcaccacctgcgtgctagtcagtcactcagacattttagttataaggtagcttatatttactccttattttatttaataatggcctcatagcaaggcggtaatgatactggtaatttgggtttgcttaagaggagccatgaagtagttttaaatgaaaaggtgaaaattcccactatagtttggagggggaggctatactggtactactacgattcacggtaagactaaatcttctgtgaaattatgaaggagaaaaagttacactggtctggtcttgctgttggattaattttatagttataaccactgtacatgataaataaccctaaaacaatgaatttgtaggtggatggcataatctgaaaaccatgttctgagcagttgatggcagcaggctgtgctggaagtgttaggcatatttatagatttcagcccaagttctgaagaggctggagagatggctcagtggttaagagtgcttgctattgcagaggacctaggttcctctacaggcaccaggcaagcgtgggacacactgagatacatacagacaaaacataaaattaaataaattgtgcataataatactagtaatatatgagtaaaataaggataaatacacatcataattaaataaataaattgtaaagttccctagaagtgagggtcaccaagccattcacaagatggctgcgctgatgcagggatatatgtgaactagaaaaaggtcaaacttaacagagaagttccaaggcatgctactgcaggcttggctagcatgcttgacctgcagaaatgctgacggccactgggaggttttcacaaatgaggaattagaagaactttttttactaatctccagaaaaaaaaaagggaagaagaaactgaagcagcctgtgatgtggaccagaaacgcagtgacagtaacatgtgtgacattgcaaaggcatgaaaggacagagctgtggaatacagacctcaggtggagctcagcatagagtcattcggggattatgcctgctgcagcaacaaaaggatgagctcaaaagagacaccgacttctgaatgcagtgggtgtttgttttgttttgtttcaaatgaattgggcagaaaactttccagctgtggaagcttctgaaccgtcccttgctgctgacatctaagcgtccgctgtgtcccagctcagtgatctagggtcttccaaacagatggtccggtgctgagcactttgaatctcaatcctgagtttctaccacgcctttggccatttaattcccagataaaagacacatacaacctttatatttataataaaccttagtcagcacaagagctgagcaaatatctgtcctctatgctattatatctattacccagccaataaccccattctataatttgctgtgcttcatctgggctgctcttaacttcagtcagccagcccacgtggccattattttaagatttttttaccccatagtgtcttctcactttactttacatttttctctctctcctcatggttctcctctgaccccaagcctaggaaccctaaaccccacccatgtctcttctgcccatctattggctgtaggcatctttattcaccaatcaggataacttggaggcaaggttaagtagtctcctgggtctaggtgctgtctctgggagcaaccagtatttagcatagcaaaagaccagacctccacaatgatcactctgaccatcggggcagaaggcacctactagcctgtgccactcacctcactttgttgaatcacatcttatcctgtagtgtgtatcactgcctgttatcacaggaaaaagtgagtcccatcaaataagatgtttcagaaagagaccatgttcatataattatcattctggtaagcttttaatggttatattttgttattaatctctttgttcctattttgcaaattataccttacagtaaatatatatgcatccaatggggtctttgaattcctccccggggagtaggaggactctttgaggatgggctgcatttaaagctaaacaacgcaacatgacctttagtccttatagatagcctagagatgagactaaataaaagaaatggtatataatgctttaagtttcccaatcagcttaaaagcttttcctataaatctttaagattatgctctggggctcaatactgcttcaagaagggcttttcttttgtatttagaattattcacctttttaaacaaaaggagaaaatggaatagaaatatgtttgcaacataattttatgactatgtgtttatttcgcgtgttctgtgggcctgcagtttgctgctgttaatgaggacaacagtggcaccaatacagtttccactcagattacattctctgttccctttctgaaagctgccctctccactgggcccaaaagagtcagtatcttaaacaagctgtacaacttagataaccatggtctcttcagactagttaattgacatatattaaaaagtaaatagtaccaaagtgaatttctgaaattaaaaatgaacatttaaaaactctaggtaaactattccttagagttaagtgttttgccaagttctgtaatcataatatgatagaaacgctcactcagcattctaaatatagaagttactccttcgcatgacactctaattcttgataaggtggagaaagagagagagagagggggagagacagaaaatatggtggttcaaggaccatttgagggaattagttatgttcttccgtcctctgtggatcttaggggttgaatacagtcattgagctcggtggatggctgtcctgttgaaaggtctgcccagcagagcaaatagacttttttatttacatggacatccgtttgtgactaatctaatgttcactcccaaagtaatcacacagacagagaggtagcttccttcagtactcttaccttacatgaatcctaccattttgttattttttttccactttaaatctttgattatgtgtttttaattagaaaatttgcatacaaatttccatacagtatgtagaattgactgtgtttgaatgggtgaagatccacatgtgtaaccctagctctggactggctctgagcttgtttgctcttctcttttgtgttctgagtaactgaaactctttcattttagcagcttagtatgcgcccttcacattgctgtgctgcctgctgcactaacattactcctttgcttatgttccccttcctgattcagtgtcattttaagcagtagtactggacctcagtaccttagccggagctcactgaggtgacagggctgaggctctgctgctgtcttttgagcttacctctttttaatgttttatggtatttctgctgccaggtttgggggttttgttttgttttgttttttgttttttgttttttttaattttctaggaacacctagaaaacacaaactaggaaacttaaaagagcagcgtcttgttccctgcgttctagaaagtccaagcctaatgccagtgtcatggttgtcaggaacatgagcctctgaaggcttcttgggaaacctttcttgtctcaacacctctggtggcaagcagtagtccatggtactctctctgtccacggtcagcatcccagtccctgccctttatctttgtgcagccgaccagctttgctttagtctgtctccttctcaggtctccttccccgctcctcttaagcacagcagtcattggattagagcccatccttccctcggatggcccatttgacctaattttacgtatttgtaactaaggtcccatttacttacacagggccctccccttcctgttttgttctttagctgaaatggtttggagaccaaatatccaatcattacaattgtgcacaagctatgttcatttggaggtaataaaggctcattctttgcttctattggtatgtgacatttttctaagtcacttggggtttgatagatatctttaaatggctgaacctgatcactgttcttttgtatgtccctgtttagctattgcaagcgttcggataatgtgagacctggaatgcagtgagacctgggatgcagggatgtcgactatctgccccccaccatctcctgctgttgccaagacagagattgctttaagtggtgaatcacccttgttggcggctacctttgcttactgggataatattcttggtcctagagtaaggcatatttgggctccaaagacagaccaagtgcttctcagtgatggagaaataacttttcttgccaaccacactctaaatggagaaattcttcgaaatgcagagagtggggctatagatgtaaaattttttgtcttatctgaaaaaggggtaattattgtttcattaatcttcgacggaaactggaatggagatcggagcacttatggactatcaattatactgccgcagacagagctgagcttctacctcccacttcacagagtgtgtgttgacaggctaacacacattattcgaaaaggaagaatatggatgcataaggtaaggggcttttgagcttgatcatggtagcctggccaatgaaagtttttttctggtacagttacacttaagttttggaaattatatgctgctaacaccagacagctgttatgttgtgtctcctgggcacagaaagccctgctctcatgcctggggtcttcacagtcctaatggaaagtaagatcttataaacattgtgtctgagtttgttctggaagctgtgactctaccttcttgttttcctttccctgtgtgactttgtcctttgcttacaacagtgcaaaagtataaatattctcagattttgataagctgtcagccacacagccttagtaactaagctgctgtcccacgctcccagttctgtataacgaggatggaccaattagattctaaggagttattcctttcaatttgcaaatttagctaaaggaaatattgttttctcctgatatttacattgcttttcattttcagcatatctaaagaacaaacctaattctccttcctactttctagtttaatataatcctaaaaatccattaaaacatgactaattctataaggcctctaacctacaaagggaagtagcattttgaaaagaatagttttctctattatacctattcatgcagacttccttccttatttctgacatacttaacaaaaatcatttagattcaaacagtttagctgcaggtgatattacagacaagtaatcccagtgctctatctagtctgaggcaaaaggatttgagctcagtgccagcctgttctatctacctggtgagttccagtcccataaataaacaaactaaaacaaccgttcctctgttcctcagatgcgagtcgatcttgtttgatttaaatagtgtgtaattattttcttttgaagctgcaggtgttatgtgggctgttttagactaaattctctctttactgtggagtaaagggtgctgtgattgtatttcatgttctctgcgagagcttgaacttgttgggctaatcgcttgtctccatcctgtctccccacctgcgtaaaaagtattttcctgtgagctgtacatgatagagcatatctacattgaaaaatgaacgagcatcaaaatggatttgttaaagtaaattttctttttcttaggaaagacaagaaaatgtccagaaaattgtcttggaaggcacagagaggatggaagatcaggtacagtgcatatcacatgctgcctgtggcaggtcctctttgcttatgtcggtataaagttggtgggtacttctggtaaggacctgaggatacattcatttgacggaaggagcctgaaaatgagtattcttgttaagctgtatagaatgaactgaataaaaatttctgcagcctaagtttgaattttaaaaaaatttaattacatctacaaattagtatttggccaccctttttcaatcagcaagaatatgtttgaggtcatttatttgtagtaaaattgcatgcagtttatttattttattgaaaataggttttttaaactatattttctgattatggttttccctcctctgaatcctcctagaacctccacctacccaaatctatatctgttctttctctctctcattaggatacaatcaggcatgtaaaataatagtagtagtagtagtaataataatgtaaaataagttaaagtaaaaacaaaccagagtaggacaacataaatagaagtagaaaagagccaaataagaaattcaagaaacacatatagacacagacacaatatttgcatacacagaaattgcataaaaccgcaagactggaaaccataatatgtatgtaaggtggagtgggaagccctgacagcacagtgagtaaagcactttcaaaaacaccactgactttgtgttgtgttgcctgtctgctgggcatgaggcctggccttagagagtggtgtgtatacccaggaagacttacataaacacttagcttttcatttgtgacctgatagcaattggaaatagtgtctgggctaggcattccggcttattgccacttcccctcagcactgaggccccatctgaatcggatccgtgcaacccttgtgcatatgcagttttaaaagttatcccttctgcaactatgctcacaggagttgccgtcttaagggagtgagcacacccctgaggcatggctccaggggtgcagagccagccataggcacagttttttttaaaaggtttatgttgtagttttgaaactcaaatttatgtgtatttgtggcagattgtttgaatgttgaaatttgccagtaacatcttttatcttcttccctttagcctggcatgccacccaccctcatttgtccttgtcaaactccagtaattaaacatggctatgtggccttttctctcattttccttagcatggctaaggagaatgggacttaaaaaataataatcatcattttaagtatgtctgagggtttgaggatatagtggtagaatatctgcctagcttccatagcttgatcctacatttgatccctggcaaaacacacacacacacacatatacacacacataaaatgacttttataaagttagtgtgctgtgctgtgatgaacagtgccataggaaatattcttggaaaagacctgaaactaaatgctctaaaaggtctaatctttacttgcttgctgatcgttaagcagagtctccaagtataaagtcactttcaccaacctctgcactggatttctggagtaattagggagagtcatttcaatataagaaaatttagtaccaaataaaattttcattcagtgaaattttgtttttgaaagtaagagcccactgtggtggtttgaatatgcttggcccagggagtgtcctgtaagatttttgttgttgttgaactccattgagacttatgttgacaataaatgcctgagagtccatgtctaaaatgctgtacctgtctgaacccaacggagataaaacttaccatttctgaaaaggatgaggtgttttatttacatagctgatgtaatgtgcttgcaacagctctattatgaatcttaatactacttcagtatatcacagcacttcaggaaatttaacatacattgtttaattccatgtcttaattgtatttgtaaacagacatttcagcagttactctaaaaagtagaaataatgagtggttgcttctggtcattaggatgaaatattgaaatgataaaattttctgggctggagagatggctcagaggttaagagcactgactgctcttccagagatcctgagttcaattcccagcaaccacatggtagctcacaaccatctgtaatggggatctgatgccctcttctggtgtgtctgaagacaactacagtgaactcatacaaataaaaataaataaatctttttttaaaaatctatatctgcataggcatttctagattaggataaattttccaaaggaaataagcacctccatgataagggcattggaaatgaagcccccgcccccacccccggtctgcacgtgtgttgaggatgagatctagggcctccttatacatgccaggcagctgttctgtcaccaagtggaatataatcctcaacccttaatttgaggttctaactttaaaatagatgtgaggggtttaaataatcatttcatgaaacttaaatgagcaagtttattactgaggtgagtataagtaattgataattttaaatatatttagctgagattgatagacacttggcaatgtcagcatcttatttaggtgatcataaactgatgggagaaatggtaaatgttagggggtgtcgctcatgtcacacaccgcagttatgctgcaaacaagatgccgggaaatagaaattcaaggtcttgttttgcgggtgcagactcttctgtctcactgattctatgtggtaacttcagtatgcatttggatagattatgtcccattttgaatgtggaagctggctgttgagaggagacttcctggtgaattcctttttctaagcattaccatctgtcttagtcagggtttctattcctgcacaaacattatgaccaagaagcacttggggaggaaagggtttattcagcttacacttccacactgctgttcatcaccaaggaagtcaggactggaacttaagcaggtcaggaagcaggagctgatgcagaggccacggagggatgttctttactggcttgcttccctggcttgctcagcctgctgtcttatagaacccaagactactagcctagggatggcaccacccacaatgggccctccccccttgatcactaattgagaaaatgccccacagctggatctcatggaggcatttcctcaactgaaactcctttctctgtgataactccagcctgtgtcaagttgacacacaaaaccagccagtacaacatcttttcacatttaatttttctcactttaaacgtggcctttaacaagcgcttataaaaatgcttaagcttaaatgttatttaagcttaatatacttaatatacagcactgtagcttaaatgttgcatgtgagagtatatgataagccatgctcaccaaggaaaagaagcttaaagagcataaaaaccctgacagcggtttctgagtgggaggctcggggactgtgctgagcaattccaaccaagggtgttttactctctgcctccatttgaaatgtttttcctgcacaacctacccaccctgtgatttcgttcactcgattatgtttgatctagggtcagagtatcattcccatgcttactggggaagtcattcctgtaatggagctgcttgcatctatgaaatcccacagtgttcctgaagacattgatgtaagtgtcatgtatcttttatgggttcccttgagtggtgagtgggtggatgtgtggtgcatgtgcgtgtgtgtgcttgcatactgggaattgaacccaagtcctcaggaagagcagccggtgctcttaagcactgagccatctcttcagaacctcttccaccagtttctttgaccatttgttgagaatattccagtcacacattttccgtgagtaaatctctctaatgctgatttgtcattaagctcagtctcctaattctgatagctaagaagggtaaattattaaaaagtgccctttactcttcctggccaattcccctttgttcttctgaaaagtgcatagacagcatcactttatagatcaccttgatgctcgtgagagggctggctcgtgctggctctagacttcggcacacttattaagagttctcccaacactgtaaacagactaatttttatattgtgcattttagatagctgatacagtgctcaatgatgatgacattggtgacagctgtcacgaaggctttcttctcaagtaagaattttacttctttttctgaatgctaagtaaagcagattaaaaatcttaatgctcacccatgacaagatttacagggaaaagatggtagaaaacctacttcctccaattatttagggtcaacatggcacatttgagcttacacgtgttgttctcacccatacaacagtggcatatctgacattactcttcccacagtctaaaaaggcagagtttccgtagtacccagggaagttctggtctgtgtttgggtctggtttcttctttcaattctcactaagtataacccttaggaatctatcaagttgagttgcattttaaattcctgtgaattcttcaggtctagaaatggaaatcattcatattttagactgacatttttcatcttcttgtgtaatttaacatttaagaacttgagctctaatatcagactgtctaggttacaactgggaaaacttggtgaagctacccaaagctgaacctccattttcttacctgtgaaatgtgaacagtgataacagctagtttcttgggtccttgtaggcaccaaatgacaggataatataaagcacctaggacagtggagccaatgagccaggagccagtgtgccctattatatctgctctaagaaagacagtaagtggaatagccaatactgactgtcttagtcaggctttctattcctgaacaaaaaacatcatgaccaagaagcaagctggggaggaaagggtttattcagcttacacttccacgttgctgttcctcaccaaaggaagtcaggactggaactcagatcaggaaacaggagcagatgcagaggccatggaggaatgttacttactagcttgctttcttatagaccccaagactaccagcccagagatggtcccacccacaagggaccctgcccccttgatcactaattgagaaaatgccccacagctggatctcatggaggcatttccccaactgaaactcctttctctatgataactccagcctgtttcaagttgacacaaaaccagccagtacgctgaccgagcagctgtgtgttcctctgcagggctgtgttctctgtttgtccctcatctcctgttgtagtctcctttacagttacagactgtcatcagtaacgagagagaagtgaataggattttgttaaagtgtttacttctatgtcacattcccttcctataataagctcacagtgaaataccaggtgaccgtgcttaacggcatctattacctaactggggtatctttttccttaaaatggatttaattttatgtgtgtttgaatacctgcatatgtgtatgtacaccatatttatgtatgcctggtacctgaaaaagggaaaagagggctttggctttcttgaaactagatggttgtgagtctccatgtgggttctggattgtctctgcaagagcggcaggcacactttagcagtgagccgctcctgtcccgagttgtcttaagacctgtgaaaggtccctaaaaaatgcagggttttacccgaataaaagatgacatcatgcagatggctttggtgttcatcaagctcttgtgtgttgtcctaaccttgctgggctttgtcgttgtgaagctgtaactccgtcaatgttttccttacctacagtgccatcagctcacacctgcagacctgtggctgttccgttgtagttggcagcagtgcagagaaagtaaataaggtaattcgttctacagttgaacatgatctgacttttatcatcactagcatatcatacattatcatctaaacagtaggctgcaattgaaataaccccatagtataaggaagcaatgtaattttaccaaatttctctgacaccctctagcagaactgactctaatagaatgagtaagaattcaattaccaaattaattttgatactcttttttatttttgttattacttttttattttattttaattaggtattttcttcatttacatttccaatgctatcccaaaagtttcccataccctcccacccactcccactcccctatccacccactcccctttggccttggcgttcacctgtactgagacatataaaatttgcaagaccaatgggcctctctttccaatgatggccaactagaccatcttctgatacatatgcagctagagacacgagctccagggggtactggttagttcatattgttgttccacctaaagggttgcagacccctttagctccttaggtactttctctagctcctccattgggggccctgtgatccatccaatagctgactgtgagcatccacttctctgtttgctaggccccagcatagcctcacaagagacagctatatcagggtccttttagcaaaatcttgctagtgtgtgcaatggtgtcagcgtttggaagctgattatgagatggatccccaggatggcagtatctagatcgtccatcctttcgtctcagttccaaactttgtctctgtaactccttccatgggtgttttgttcccaattctaagaagggacaaagtgtccacactttggttttcattcttcttgaatttcatgtgttttgcaaattgtatcttatatcttgggtatcctaagtttctgggctaatatccacttatcagtgagtacatattgtgtgagttcctttgtgattgggttacctcactcaggatgatgccctccaagtccatccatttgcctaggaatttcataaattcattctttttaatagctgagtagtactccattgtataaatgtaccacattttctgtatccattcctctgttgaaggacatctgggttctttccagcttctggctattataaataaggctgctatgaacatagtggagcatgtgaccttcttaccggttggaacatcttctggatatatgcccaggagaggtattgtgggatcctccggtagtactatgtccaattttctgaggaacggccagactgatttccagagtggttgtacaagcttgcaattccacgaacaatggaggagtattcctatttctccacatcctcgccagcatctgctgtcacctgaatttttcatcgtagccattctgactggtgtgaggtggaatctcagggttgttttgatttgcatttacctgatgattaaggatgctgagtttttttttcaggtgcttctctgccattcggtattcctcaggtgagaattcttggtttagctctgagccccatttttaatggggttatttgattttctggagtccaccttcttgagttctttatatatattggatattagtcccctatctgatttaggataggtaaagatcctttccaaatctgttggtgacctttttgtcttattgatggtgtcttttgccttacagaagctttgcaattttatgaggtaccatttgtcgattctcgctcttacagcacaagccattgatgttctattcaggaatttttcccctgagccaatatcttcgaggctgttccccactctctcctctataagcttcactgtctctggttttatgtggagttccttgatccacatggatttgacattagtacaaggaaataggaatggattaatttgcattcttctacatgatatccgccagttgtgctagcaccatttgttgaaaatgcttttttccactggatggttttagctcccttgtcaaagatcaagtgaccataggtgtgtgggttcatttctgggtcttcaattctattccattggtctacttgtctgtatataccactaccatgcagtttttatcacaattgccctgtagtacagctttaggtcaggcatggtgattccaccagaggatcttttatccttgagaagagtttttgctatcctaggttttttgttattccagatgaatttgcatattgccctttctaattcgttgaagaattgagttggaattttgatggggattgcattgaatctgtagattgcttttggcaagatagccatttttacaatgttgatcctgccaatccatgagcatgggagatctttccatcttctgagatcttctttaatttctttcttcagagactttaagttcttgtcatacagatctttcacttccttagagtcacgccaaggtattttatattatttgtgactattgagaagggtgttgttttcctaatttctttctcagcctgtttatcctttgtatagagaaaggccattacttgtttgagttaattttatatccagctacttcattgaagctgtttatcagatttaggagttctctggtggaattcttagggtcacttatatatactaccatatcatctgcaaaaagtgatattttgacttcttcctttccaatttgtatccccttgatctcctcttgttatcgaattgctctggctaagacttcaagtacagtgttgaatagggaggaagaaagtggacagccttgtctagtccctgattttagtggggttgcttccagcttctcaccatttactttgatgttggctactggtttgctgtagattgcttttatcatgtttaggtatgggccttgaattcctgatctttccaagacttttatcatgaatgggtgttggattttgacaaatgctttctcctcatctaacgagatgatcatgtggtttttgtctttgagtttatataatggattacattgatggatttccgtatattgaaccatctctgcatccctggaataaaacctacttggtcaggatggatgattgttttgatgagttcttggattcagttagtgagaattttactgagtatttttgcatcaatattcataagggaaattggtctgaagttctctatctttgttggttctttctgtggtttaggtatcagagtaattgtggcttcatagaatgagttgggtagagtaccttctgcttctgttttgtggaatagtttgtgaagaactggaattagatcttctttgaaggtctgatagaactctgcactaaacccatctggtcctgggattttttttggttgggagactattaatgactgcttctatttctttaggggatataggactgtttagatcattaacctgatcttgatttaactttggtacctggtatctgtctagaaacttgtccatttcatccaggttctccagttttgttgagtatagccttttgtagaaggatctgatggtgttttggatttcttcaggatctgttgttatgtctcccttttcatttctgattttgttaattagaatactttccctgtggcctctagtgagtctggctaagggtttatctatcttgttgattttctctaagaaccagctccttgattggttgattctttgaatagttcttcttgtttccacttggttgatttcacccctgagtttgattgtttcctgccgtctactcctcttgggtgaatttgcttctttttgttctagagcttttaggtgtgttgtcaagctgctaatgtgtgctctctctagtttccttttggaggcactcagagctatgagttttccccttagaaatgctatcattgtgtcccataagtttgggtatgtagtggcttcattttcattaaactccaaaaagtccttaatttctttcttcattccttccttgaccaaggtatcattgagaagactgttgttcagtttccacgtgaatgttggctttctattatttattttgttattgaagatcagccttagtccatggtgatctgataggatgcatgggacaatttcaatatttttgtatatgttgaggcttgtttttctgaccaattatgtggtcaattttggagaaggtaccatgaggtgctgagaagaaggtatatccttttgttttaggataaaatgttctgtagatatctgtcagatccatttgtttcattacttctgttagtttcactgtgtccctgtttagtttctgtttccacgatctgtccattggtgaaagtggccatctttatagtcactgaagacatacaaatacatattcatatcaactggaacaaacctaatttctttttaaatgttttacatggaaataagttaggggttgttatttgcattacaaagttactcatccctttccttcttttctttttttttttttttttttttttgagaacaagcctgtgtacttatatgaactttaatttgccaaattcataattcttattcaatcatttatgacagaatgctaaaactctcattatattttagctaggcatttagagctgttatgtgtaaccccaaaaagtagctttccacttgagatgctgaaggccttgggttccgtgggctgtcatcatggttggctgtatgaaaagagaaaggctccattgtttgggcatcacttaaatattttttcacctttcatcttcttttaggttaagtagcttgtccttgatcatttcatttttgagagacaacttgccactactctagttgaaaagtgctgtcttgacgctgtctctggctgtggtcagagtccagcagagctgcacagctggttacctttctctgtacagctctaggccaactcttcttactggcgaccatttctaaatccaccattcacttgttccccatgaaagtgagtagggttttttctgtggaagattttgggcagtcctgttgccactttgcatcagacaatagttccctcattgaaacacgcagtttattctccagagcggtctgcccactccaaaggcagtaggtgctgggtagagatatgccaagtatcacactaggctatgactgctcactcagatcactcggatgaagctttcatggccaaatacagttgagaaagaacaaatattcttcacttagagagcaacaagagttattcaagtgtaacaagttctgagattccatgcagttgatttaccagctacttcctaaacttaactggccacaaaatccctttgtaagcagtatgttgttttgacccatgccctgtcaaaggatactccttacttgggaactgttttaatgatggcaacaaaaatttctatttaaatttatttcataagcaagcaaagatctttttacttcacattccaatgttgactcttttcctctagatagtaagaacgctgtgcctttttctgacaccagcagagaggaaatgctccaggctgtgtgaagcagaatcgtcctttaagtacgaatcgggactctttgtgcaaggcttgctaaaggtacacttgccgatcatttatcatgtgtgacgcaacaagtagagatggagggtacaaataatcactgagaggctttggaaagtatattgttagcatttaatgtctcatagttttagttgtctgggtactggtttgttttcatcattctgagcatgaagtgtatgtcttagggatttatagttcgtatcatgtatgaaacaccatggggtaatatttatatttcacttggttccctctagctatgtgtctggccccagtgctttccttgtaaatgcatgcttgaatcagactgagctgatatgataatgttgatgctccttttgcttactgagtggctatgaatatgcaccatacttactcattgtaagaaattaaaatgtctcttaaggatgtaaacatagcaaaatgaagcaaaacaaaagcgatgctgttttaggtaccctaactgaccttgtgtattcaaggagcattcctacttctgtgatgcaaaagctgtctacactgggcagatctacaaccagcattaaaccaaatagggaatcactgaaatcacgttatcaaagatgagaaacaagataataatgtctactttcacggcttttattcaggtctagtgctataagtttttgccaaaacaaaaatgaaaacatagactctgggctgaggctttcccttagcagaaaagtgcttacttgttgtgtccggccagcagatcacagcctgggttctagcctggaaaggcattttggaaacctggaagagaagaggggctaggtaacgagagaaagaacggagccaagtcaaaagcaactctgatcaaagctcaattttactatatcagcacgcagttataaaggaggggaagggggggccaatagcaaggcggcaggttccagcagtgggcgtggcagaccgattgagccggcaagctccttccaggtgtaaacagtggagccctaaggctgggggaggggaggctacacttagcatgcctgatgccctagatgccacctaaatgacaaatccagtccagtacaggatgtagagcacccccccccaaaaaattattttttttgtataccagaaatgaaattgctgagaaaaaaaaatgaagaccataattatactcccagtagctacaaactaaacagccccatagatgaagtgagtgatgtctgctgtgacaattatgaaatgaaagaagtaaagatgaacaaatgaagggaagacatccagtactcaggactgaaagactgctgctaaaatgcctatccaacccagagctctctgcagactctggacagatccgctctagatgtgaagatggtcttttttttttttttttttttttggtttttcgagacagggtttctctgtgtagccctggctgtccaggaactcactctgtagaccaggctggcctcgaactcagaaatccgcctgcctctgcctcccgagtgctgggattaaaggcgtgcaccaccatacctggctttttgtgaagatgttcttaacagaactagaaagaagtaccccttggtttgctgcccttctgatgcagtatccccaaaggctcgcatgcactgaacatttcatcttacctggtgccactgttgggaagtgatggaaatgcgaggaattgtagcctcgttgagatgtttctcattaaggcactgggggcatacctatggagcatacagtaggaacctggtttgcaacctctcccctctccatccaggctctcccctgtgcacctggccttggtgttctgccactccatgaacccaaagtaaagtggactatgcccttagactgtaacagtgagtcagaagaaacatttcctctttaaagctgagttttctgggtgctttgtcatgttaatggagtctgattagtacagaccctgagtaggcagggcaatcttatgcagaaacatcaaagctggtagcatagacatacctaatttcacaatagacactgatggactcagtctggagtacttacagtaagaatatacagcagagatacggagctctcttacagtggtgctctgggagaactggccgtcctgtgaagaaaagccagagtggctcattctcaccagacacaaactgagctcataagacgcttgaacctgagatcctggtcagcagccactagaagaaaacttaggagaaaccattcaacacgtcagtctggggaaaagggtggttttggttttggttttggttttttagtatattccccaaatcaaaaacaacaaaacccaaacttgacagatgacatcacactgcaaagcttttgcacaaccaagaaagcaacctgcagagtgcagtaataacccacagaaggagaggagatacttgtgggcagttcatcacacaggtcaatataagcaagtactgatagtgtggccatctccaaagaagatatgaaaataactggtatatatgaagtagtacttagcattgctgcgtatatggtaaattcaaaaccatgatgagatattgccccacttagatggatattatcaaaacaacatcaaaaagtgacaaatgctttcaaggatatggggaaagtgtacttgcaggaatttaaattattaatttgccattcaagaggataggatggcagtttaaattaaaaaactagaagtggtagagcagtcgcctagaacatacaaggttcagcactataataaatgagcaattagacatttgaagcaacaatctcaccactaggcaagtcctaaaagaaatggactcgcttcttcttcttcgggaaaacaccaaatggcagatgacgccggtgcagcgggagggcccagaggacctgggggctcaggattaggaggccgcggcggcttccacggaggattcggcagcggtcttaggggccgtggtcgtggccgaggccgtggccgtggtcgaggccgcggggctcgtggaggtaaagctgaagacaaggagtggatccccgtcaccaagctgggccgcctggttaaggacatgaagatcaagtccttggaggagatctacctgttctccctgcgcattaaggagtctgagatcattgatttcttcctgggtgcgtccctaaaggatgaggttctgaaaatcatgccagtgcagaagcagactcgggctggccagcggaccaggttcaaggctttcgtcgctattggggactacaatggtcacgttggtcttggtgttaagtgctccaaggaggttgctactgccatccgaggggccatcatcttggccaagctttccatcgtccctgtgcggagaggctactgggggaacaagattggcaagccccacactgttccatgcaaggtgacaggccgctgtggctctgtgctggtgcgtctcatccctgcccccagaggcactggcattgtctctgctcctgaagctcctgatgatggccggtatagatgactgctacacttcagccagaggctgcactgccaccctgggcaactttgctaaggccacctttgatgccatctccaagacttacagctacctgacccccgacctctggaaagagactgtcttcaccaagtctccttatcaggaattcacggatcatcttgtgaaaacccacaccagagtctctgttcagaggacccaggctccagctgtggctaccacataagggtttttatatgagaaaaataaaagaattaagtctgctgaaaaaaaaaaaaaaagaaagaaagaaagaaaagaaatggactcggtatgtggatgaagcccaggcaccttcatctgtgttgcagcacgagtcaccatgcaggatcagtctaaacgcccatgcacaaatgaatggtacatagccacagtgaagtgtttgaccacaaaaaggaaagtcagttgtgataagtgaaacaagccaggcacagaaagataaatgctgcatgttatcattatgtgtaaaggctaaaacgtttatctcatacaagtagaaggtaaatacggagactaccagaacttataaagagttctaggaaaaagctatagagaggctcagggttgaataactaaaattatacctaaaataactaaaaggatagcttacaatattctgtagcactgtagaataattgtgacagtttgttgtatttttctggtttgtgtatgtgggagagaaagtatgtggacagaggttgatatcaagtgtctgactctgcactgcattattttaggcagggtctctctctaaccattgaatggactggctaggcagtggtgccctaacatctacctgtccgtacatctcccaatactaggttataagtacactgggttttaagtacaggctataggtatagatataggctacaggtatagatataggctgctgcaactgattacatgggtgctgggaacctaacataggttgggtcctcatgtttacacagaaatcagtactgtgcctactgagtcatttccccagttctagtatttgttttttaaatagctagtaattggaattgtgaatgttcctaacaaaagaaaatgataactatctgagatgctagttatgataccctgagtgaatcacactttgtgtgcatgtactgaaattcattgtaccctgaaaatacaaaaattgctctgtgttgattggctagatgcatgtgtattagtcagcaatctctagagtaataaaacttagatatatgggatgtattagacttttggccttacaggccaagatccagctaatccatcagtggcaggctgtgaacagtaagtctaagaatccaatagttgttcagtccacaaggccgggtggctcagctgccttctgtatacagtggaatcccaaagaaataggcgccaaagctagtgaggaatggtcttgctagcaaagcgaaggtgaaggtaatcaggcagaagacaagaccttcctttttccgtgtccttatataggctcctagcagaacaagtggcccagactagatgtggattaaatgttttgggtttggtttggtttgatttggtttggtttggtttggtttggtttggtttggtttggtttggtttggcttttcgagacagggtttctctgtatagccctggctgtcctgggttgtagaccaggctggcctcaaactcagaaatctcttgcctctgcttcccaagtgctgggattaaaggcgtgcacaccactacgcccggctcaatagcattaaatggcatgtcttttcctatctcaaatgatctggattaaaagagtgtcttcctacctcaaaggtctggattagaagtggatctttctacttcagattaagttaaactctctcacaggtgtgccctctacttttggatttttggttctagatggagtcaacatgacaaccaaaagtaactattacaagtccacccaatatcaacttgatacacaatcatatctccttatgtcataattaatttccaaatgaaaacaataaccatgtcataaaaacacctaaacatgaataactattccacatacaatcagaaatgcattcattatatatttaaccaagtcctaattatgcctaacgtgatataactattcttcatacaacagcaaacatgataaatttacaataggtggcaatgtcttattcttttaatatctcaaacttaaatatgataaccattgatgttatcttaattgatgttatatcatatgataaagaaattgatgaaagaaagcacaaatgtctgtataaatgctttcttaagaaaataggacagaaactctgtcaattataatcatcttttctgcaactagtcatgtggccttagtatttataactaccttcctctgctaaaccattttgtattttctccacccttggcaagaacctcagcaggtcttggctcttttcctggaggagtgacccataccttcattccttacatgtatgtgccctttgtcatcctgcctggaccaggttgttgtaacattgactttaatcacaggacatcgtagcaccaacacatgccccaaaggatctcctgccctatagacataacctttcttacctccatagtggggaggcagtcccagtcctccttggtagtctgcatcagtcacgcctcctaacactgttattcctttcttagccggttgacttaagggcatcagaaggccaaagttgccagaggaaaatctgagcttccagttcaatgaatgtaatgttgttctaggcaagcagaactgaaggtctcaggaataggaagcaaacacttcccatggatcactacagggtgagagtgagtagaattattctcttttctaccacttgactcctggacctatggatcctggtatcaaagaaaatgtctcatatattgtacactgattcagagcatgccttctggaaaaccctgccccagcccttcatactgctgccatcaaattgtcacctgtgtcttcctggtaccaacttttgtcctggttagggttactattgctgtgaggaaacaccatgagcaccaaagcaacttggggagaaatgggtttattcagcttatgcgtctacatcacagctcatcatcaaaggaagtcagaacaggagctcaagcagggcaggaatctggaggccgtggaggaaagctgctgactggctcgctccctaggcttgctcagactgcttatagaactcaggaccaccagctccagggtggccccaccccgcaatggattgggccctccctcaggaatcacaattgccccacagacttacctacagcctaggcattttggaggctttgagtctgcctcctctctgatgattctagcttttgtcaagttgaagcaaaagtagacaggccttaaactcacaacaacccacctgcctcaattttctgagtgctaatattatatcaatttaaaatttaaatataacatataaagggcaatagaaaggactagattcatgtaatggatacaagttatggaagatgtgtgtgtgtgtgtctgtctgtctgtgtgtgtgtttctagtttaattctgtcatgatttttttcttgtaggtggtaggtgagtgcatggaatacatttgatactgaaagggtaaattgaatgtggagcctcacagcttctgttccacatgcctatgataaccgtagaaattcatggattagtatagacgttgagtctggttaattttggtgtgtgatatttatatatatatgtatatatatatgtgtgtgtatgtatgtatgtatgtatatatatatatgtatatgtgtatatatatatatatatatatatatatgcaagatttcttataattaagtttacaaaattaaaaactatcttaaaaattgaattcttgcaaataaaaatttagcttttggtgattggattcttaatatggttgatgtttacctagaaagttaaaagccctgagttcagtctccactttcacccccaaaatgaaaatcagcttttgggtttcagatcatgagctcagaattaaagaaaacacatttctaactttgcttttacaaatcttaattttaccaatttcctttaaagtcacaatgagatacacagtacttcctagcaccccttgttcaattagataatgtgatttctgaaagagctccctctacacagggcacagggcaggtgcaaaactgtgattgggtgaaatacctgcgagctctccaagcaaagccaggcctatttgctttagctgccacatcgggttcttagacccgacatcccttcccacctgtatcctccctaattccttccaaccccacaacactaggtaggagagaaagaaggttagtggtggaagtttgcacacatctttttagactatttcctactgattaggggtgttaggtccttgagacaagtccagtcttcattgtcaggatatctccaacttcttcttctcatctctttgctcacaaagtttatcacaagttgataaactacaacaacaggaaccagcagtagcaaggacatcagagttgtatagctttccagaaaatactttgatatacagtaattatcctagcctttaagagtgaaagatttggcagcctctgtgttctacactcagcataataccttgtatactgcaggtatttgctgcatggtaagtggctgcccagctacctagaaagaggtaaatacttttctattaacatacatattcatttagatataggaagaagataaaacaatggagaaaggcagtcataattttacagaccagcaagtaaacgcattaacttggcataggtctttgtagtctttttctgcagtgcgtatttcctgcagtgcccacaccctacagttggattgcacgtggcatgttctgacccactttttatggtatactgtgtactgtcactgtcaacacaaatggtagtggctggatttttatacagtatcagcttgaaggttatttctgaacaagccctgtaccagattcacaggaatatgcatctcttatcattactatattcttttaacaattgcttctctcagttggcatgtggtcagtgagttctctcttccttctgacaggatgcaacaggcagttttgtcctacccttccggcaagttatgtatgccccgtaccccaccacgcacattgatgtggatgtcaacactgtcaagcagatgccaccgtgtcatgaacatatttataatcaacgcagatacatgaggtcagagctgacagccttctggagggcaacttcagaagaggacatggcgcaggacaccatcatctacacagatgagagcttcactcctgatttgtatgtgacgcttggccttaggtgtcattgttaaacaacataaaacttctcatttatgagtaaaaacagtgcaagttgtatttaaaagaaaagaaatatgacaagcacatactcaggcactttttctttattttcttaactttaaggtttttttttttttaagatttatttattattatatctaagtacactgtagctgtcttcagacacaccagaagagggcgtcagatctcattacaaatggttgtgagccaccatgtggttgctgggatttgaactcaggacctttggaagagcagtcagtgctcttacctgctgagccatcttgccacccccaactttaaattttttatactattatttttagacagtctcactgggcctaatgacttacataggtggcctggaactcactatatagatcaggctagccttcaactcccagatatccacctgcctctgccacccaaatacttggattaaaggcgtgtgcctccatacctagcctaaatcttcatttcttaaaatactgttttgctaagataggtaaagatttcctcttaaaaataaatacttagcaaatatataccgatctcctaattacttaatgaagggccagcttaatagttatcagtcagttatcagtgccagcccctactgctgggaatttagtgtataacgttcattgtatggtagactgaagtaattctaagtatttttttcttgggtgtgactatcaaacacagaaaagtatttgaaatttataaagagaacaggttttttctttgcattttatattttgctatttatttcttaccagaagatgcgagcagcaaagtaaaaggcagtaagtgctgatgggtttggaggaacttgggattttaattataaaacttcaagaaagcatttcaatggtgttctagagtctaaaaaagaatagtgagaccctattcctgttctctccgatcaaccaagagcttgaaatggtgctagtccttagtatacactgaaaagacgctaagtgtggtcatcccggttggagggctttaggaagcagtgaccctggaccaatgggtgtcaccgtgtgtctgaagaagaaagcagagctgaaacaagaggcgcatggtagggacaccagcagccacagtaaactgctgcccagaggtccctgtgtggggctgcagaattaaaagaacccattctacacagctctgctgtgctctgttagtgctgagaaaggttgagaggaattgtttcagaagaggaatcgttcaaattgaactcttatgtcactagttcacatactggcaatcttggaaaacatagaaattttctcactgagtctgcgtgcctgcgtcttcctcgtgactaatatacttgaagtcctgtttatttttttagttgattgtttagaatctcttctcaggaaatgaggtaaacttgaatggatttgcaccatgttagtgtttttgttttgaatatgtttgtttggaagatttgaagaaaaagcaattgttcagctattctggcatgacaaaatcatgtcatgaattttagaattttatttccagttctaagtaaatgttttgaatataaaattgtcagaaatattttcagccacaagattatatcttctattattgtgggctcatgatagtatcagtgtggtttaaataatattcacttttgagtctgggaggtttgaggtttcagattcagggactcacacactgggcaattactgtaccactatgcagttgcttattagtaccacagagtaattcccagttaagttacttttaattttaacctttttaagataaaagcagtctgatgatacattaaagtcggacatttccttgaagatagtctttccttttccagcttttgtgatccagatctcattcagtaaagcagaaattgggaaatagtggacttaagttctaagggacccacaaaccccgtgactgtgctgtccgttttcagccagtaaccatgaagtgctggcgtcccttccagcgcccctttctccatttggtgcactcatccctcaaggctgagaggcgtgctgctctcctgtctatttccctcttccccatggttcctgggcagtgatgttgtgatctctaccatctgagtcttgctttgcatttatcttactgtgaaaaatgttatattttccctctgacatgaatataatagcctagggaaagacagaagtaaaacactgaaagggaatgggggctgagaaaaaaacagtcattagcttctgtctggccagcatgctgaagtgggtcacctcagttggccattttgtctgaacgttacatgccagccaaccttagctgcggtagtaataagttatgctgctggctcatacttacagatggtaagtctcttgacctgaggcaaacgtgtaaggtgacggttctaaacacactgatggacaggcacatgccctgcctggatagcctcaaaacacaaacagtgtacaaatgtacccttgcgttaaagtggatctatgtgcgtttgtgtttattttctgtgcattaagtatgtatatgtatgtgtgtttatattgtgcacattgagtatatgcatgtgtgtttacactgaatactgaacccacggcctcctgcaaactaagtatgcattccaaatgcacacatctgtcttcttacacatctgtttataaaacttcaacttttttactagagcaagaagttgtggaatgtaactctgtaaaaccgtttaatatctgaacctttttcttcttaggaatattttccaagatgtcttacacagagacactctagtgaaagccttcctggatcaggtaaatatgatgccacccattgccagacaaaagaacatcatatattttcttttaaaatatgtcccacagtgcctacagaatatataaaaagcaccaaagaattaaagtgctagaggcctttctaaagtctgtaaacggattcctctttgaattattaatgggaaatagcctgtatattaaccgttaaagcagcattctccatcctagtggctgcttcaggtccaaccctctgcctttagaatttttgtggttggtgaagacaggggtgtgctttcatttgtgttaattgaattgaaaatattcttaaaacttaggttgcttctgcttaaatggtagcatccttattgtctctgtttttaaaagtatctgatgagtaaacatctggagatggtactggattctatgcgacttgtttctatacgtaagcagagctttgtcataatagcatgctgggaatcaggccaagatcctgtgccatagacatagagttgagatgaggagaacctcgtgttcactgggacttgtgggtctgggtctgtgtgaggtgaggacagcctgtaatcccaagtctctgaagctgaaaagtcccctcctctactccacacaacctgaagtcattgacttagttatttccataataaaataaggagatattttaaggtagaatacaagatctaagtgcattaaactagggaatctgaaaaggggacagtgggtttccagacatttgccgctaccagagtcttgccctttggaaatcggaagaaatggctgtaatgggtgttgtgtgtcagatcctgtcaacaatgtcgcggaagctgcactgtcttgtgtccctgcaggtcttccatttgaagcctggcctgtctctcaggagtactttccttgcacagttcctcctcattcttcacagaaaagccttgacactaatcaagtacatcgaggatgatacgtgagtcctgctcctctagaggaaagcctttatgcattgacagttgctgttcgttccctttgaacattgtctgtattataatgcgggggtttttgtctcttttgttttgtttataggcagaaggggaaaaagccctttaagtctcttcggaacctgaagatagatcttgatttaacagcagagggcgatcttaacataataatggctctagctgagaaaattaagccaggcctacactctttcatctttgggagacctttctacactagtgtacaagaacgtgatgttctaatgaccttttgaccgtgtggtttgctgtgtctgtctcttcacagtcacacctgctgttacagtgtctcagcagtgtgtgggcacatccttcctcccgagtcctgctgcaggacagggtacactacacttgtcagtagaagtctgtacctgatgtcaggtgcatcgttacagtgaatgactcttcctagaatagatgtactcttttagggccttatgtttacaattatcctaagtactattgctgtcttttaaagatatgaatgatggaatatacacttgaccataactgctgattggttttttgttttgttttgtttgttttcttggaaacttatgattcctggtttacatgtaccacactgaaaccctcgttagctttacagataaagtgtgagttgacttcctgcccctctgtgttctgtggtatgtccgattacttctgccacagctaaacattagagcatttaaagtttgcagttcctcagaaaggaacttagtctgactacagattagttcttgagagaagacactgatagggcagagctgtaggtgaaatcagttgttagcccttcctttatagacgtagtccttcagattcggtctgtacagaaatgccgaggggtcatgcatgggccctgagtatcgtgacctgtgacaagttttttgttggtttattgtagttctgtcaaagaaagtggcatttgtttttataattgttgccaacttttaaggttaattttcattatttttgagccgaattaaaatgcgcacctcctgtgcctttcccaatcttggaaaatataatttcttggcagagggtcagatttcagggcccagtcactttcatctgaccaccctttgcacggctgccgtgtgcctggcttagattagaagtccttgttaagtatgtcagagtacattcgctgataagatctttgaagagcagggaagcgtcttgcctctttcctttggtttctgcctgtactctggtgtttcccgtgtcacctgcatcataggaacagcagagaaatctgacccagtgctatttttctaggtgctactatggcaaactcaagtggtctgtttctgttcctgtaacgttcgactatctcgctagctgtgaagtactgattagtggagttctgtgcaacagcagtgtaggagtatacacaaacacaaatatgtgtttctatttaaaactgtggacttagcataaaaagggagaatatatttattttttacaaaagggataaaaatgggccccgttcctcacccaccagatttagcgagaaaaagctttctattctgaaaggtcacggtggctttggcattacaaatcagaacaacacacactgaccatgatggcttgtgaactaactgcaaggcactccgtcatggtaagcgagtaggtcccacctcctagtgtgccgctcattgctttacacagtagaatcttatttgagtgctaattgttgtctttgctgctttactgtgttgttatagaaaatgtaagctgtacagtgaataagttattgaagcatgtgtaaacactgttatatatcttttctcctagatggggaattttgaataaaatacctttgaaattctgtgtatgttttagttcattatttagggaaaacgctgctgtgaaagggggcgtgatcagcttcctattctgcgacagtcgtgttgaacggaacccattggttttcatcttcgctcccccccccttggtttttcgagacagggtttctctgtatagccctggctgtcctggacctcactctgtagaccaggctggcctcgaactcagaaatctacctgcctctgcctcccaagtgctgggaggcagttgccccaccaactagtcttcttttttcaaagaagatatttaaagctaacgaataatgctagactcttacatcttaaaaaaaaaagaagagaaaagaaaagaaaaggtaatcacactgcccagtgtgtagtgcatgcttctacttccggtccttgggagatgggggcaggatgagacgctccagaccggcttccaatacagagttcaagacccactgagctacgtgaggctacacgagcctgcctttaaaaacataaagctaaagctttcttcttaacttccagtattgcaccttgattcccccttcaaatttcacatacaaaataattcttaaattctcttttgaaaaatgttctactgaggccagagagacagttcgcttggtaaaggtgcctgttgccaaacgtgataacctgagttaaatcatagccccacatgggggaggaagaaacccccgcagcttgccctctgatgccatgtatgcactaaaacacgcacgtgtgtgcgcacacattttttaagttcctattacattgatagtaatataatttaaactgatttattctccccaagtcattgatacgggtgtccaacgtaaaatccagcggctgaacaaagcacttttaggcgctttaagttggaaagcaagaaacggagattgacactgtcactccaagagaaaactcttcgtagtagcgagatcggctgtggagtgaagatgctcagaggctgggaacgcacacagctcaggagtggatagcatcccccagcctcaactcctaacactgggaaagcgtagggctctcagatgaggaaacaaaaccatacaaagctgctgcaagctaaacagaaaaatagtggcattacactaactgttgtggaattgtacagaccgattctcctcccaatctgccgagtgtgggcggcttgagagaatgaagagagctactggcctcaggtaacagtgcttcccacaggactgtctcaggctgccaccaccataaatagcattttagacgtgacagagctaaggcttgacacacagccaaaagctactcacattccatttcatccccagctgttctgtcatcgctaagcacagagcattcagcacagctcttccctgtggtgggtactcagcactgttgagttgaaaggattgaaaaaactcaagactatgttctcaaacatttttttaagctctttttaaaaccaccttagaatgaaagcttttgacttcttattaacatgcactaacttcatatacacatttagtgttattgtacaggcacgaagcatactctggtcagaacctgtctcctttggtccaccctccccaccgttttcagcttctattccaccttccatacgtctcaagatccacatgtgagagggaacactcagagccttgtctttctgtatctgggatatctcacttaacatgatattctccagttctgttccatccatttcattgcaaagagcaagatttcactctacagccaaataacacatttgtccatgtatatccgtatttttccttattcatctgttgaatggcacaagactgatatcatgggtaatatctat

SEQ ID NO: 94 - DNA, 3435 bp, Rattus norvegicus
cgtttgtagtgtcagccatcccaattgcctgttccttctctgtgggagtggtgtctagacagtccaggcagggtatgctaggcaggtgcgttttggttgcctcagatcgcaacttgactccataacggtgaccaaagacaaaagaaggaaaccagattaaaaagaaccggacacagacccctgcagaatctggagcggccgtggttgggggcggggctacgacggggcggactcgggggcgtgggagggcggggccggggcggggcccggagccggctgcggttgcggtccctgcgccggcggtgaaggcgcagcggcggcgagtggctattgcaagcgtttggataatgtgagacctgggatgcagggatgtcgactatctgccccccaccatctcctgctgttgccaagacagagattgctttaagtggtgaatcacccttgttggcggctacctttgcttactgggataatattcttggtcctagagtaaggcacatttgggctccaaagacagaccaagtactcctcagtgatggagaaatcacttttcttgccaaccacactctgaatggagaaattcttcggaatgcggagagtggggcaatagatgtaaagttttttgtcttatctgaaaagggcgtcattattgtttcattaatcttcgacgggaactggaacggagatcggagcacttacggactatcaattatactgccgcagacggagctgagtttctacctcccactgcacagagtgtgtgttgacaggctaacgcacatcattcgaaaaggaaggatatggatgcacaaggaaagacaagaaaatgtccagaaaattgtcttggaaggcaccgagaggatggaagatcagggtcagagtatcatccctatgcttactggggaggtcatccctgtgatggagctgcttgcgtctatgagatcacacagtgttcctgaagacctcgatatagctgatacagtactcaatgatgatgacattggtgacagctgtcatgaaggctttcttctcaatgccatcagctcacatctgcagacctgcggctgttctgtggtggtaggcagcagtgcagagaaagtaaataagatagtaagaacactgtgcctttttctgacaccagcagagaggaagtgctccaggctgtgtgaagccgaatcgtcctttaaatacgaatctggactctttgtacaaggcttgctaaaggatgcgactggcagttttgtactacctttccggcaagttatgtatgccccttatcccaccacacacatcgatgtggatgtcaacactgtcaagcagatgccaccgtgtcatgaacatatttataatcaacgcagatacatgaggtcagagctgacagccttctggagggcaacttcagaagaggacatggctcaggacaccatcatctacacagatgagagcttcactcctgatttgaatattttccaagatgtcttacacagagacactctagtgaaagcctttctggatcaggtcttccatttgaagcctggcctgtctctcaggagtactttccttgcacagttcctcctcattcttcacagaaaagccttgacactaatcaagtacatagaggatgacacgcagaaggggaaaaagccctttaagtctcttcggaacctgaagatagatcttgatttaacagcagagggcgaccttaacataataatggctctagctgagaaaattaagccaggcctacactctttcatcttcgggagacctttctacactagtgtccaagaacgtgatgttctaatgactttttaaacatgtggtttgctccgtgtgtctcatgacagtcacacttgctgttacagtgtctcagcgctttggacacatccttcctccagggtcctgccgcaggacacgttacactacacttgtcagtagaggtctgtaccagatgtcaggtacatcgttgtagtgaatgtctcttttcctagactagatgtaccctcgtagggacttatgtttacaaccctcctaagtactagtgctgtcttgtaaggatacgaatgaagggatgtaaacttcaccacaactgctggttggttttgttgtttttgttttttgaaacttataattcatggtttacatgcatcacactgaaaccctagttagctttttacaggtaagctgtgagttgactgcctgtccctgtgttctctggcctgtacgatctgtggcgtgtaggatcacttttgcaacaactaaaaactaaagcactttgtttgcagttctacagaaagcaacttagtctgtctgcagattcgtttttgaaagaagacatgagaaagcggagttttaggtgaagtcagttgttggatcttcctttatagacttagtcctttagatgtggtctgtatagacatgcccaaccatcatgcatgggcactgaatatcgtgaactgtggtatgctttttgttggtttattgtacttctgtcaaagaaagtggcattggtttttataattgttgccaagttttaaggttaattttcattatttttgagccaaattaaaatgtgcacctcctgtgcctttcccaatcttggaaaatataatttcttggcagaaggtcagatttcagggcccagtcactttcgtctgacttccctttgcacagtccgccatgggcctggcttagaagttcttgtaaactatgccagagagtacattcgctgataaaatcttctttgcagagcaggagagcttcttgcctctttcctttcatttctgcctggactttggtgttctccacgttccctgcatcctaaggacagcaggagaactctgaccccagtgctatttctctaggtgctattgtggcaaactcaagcggtccgtctctgtccctgtaacgttcgtaccttgctggctgtgaagtactgactggtaaagctccgtgctacagcagtgtagggtatacacaaacacaagtaagtgttttatttaaaactgtggacttagcataaaaagggagactatatttattttttacaaaagggataaaaatggaaccctttcctcacccaccagatttagtcagaaaaaaacattctattctgaaaggtcacagtggttttgacatgacacatcagaacaacgcacactgtccatgatggcttatgaactccaagtcactccatcatggtaaatgggtagatccctccttctagtgtgccacaccattgcttcccacagtagaatcttatttaagtgctaagtgttgtctctgctggtttactctgttgttttagagaatgtaagttgtatagtgaataagttattgaagcatgtgtaaacactgttatacatcttttctcctagatggggaatttggaataaaatacctttaaaattcaaaaaaaaaaaaaaaaaaaaaaaa